(19) United States
(12) Patent Application Publication
(10) Pub. No.: US 2026/0272468 A1
(43) Pub. Date: Sep. 17, 2026

(54) Sustained Release Liposomal Pharmaceutical Embolizing Agents for Treating Chronic Subdural Hematoma, Intractable Migraines, Headaches, and Other Pain Conditions

(71) Applicant: MG Stroke Analytics Inc., Calgary (CA)

(72) Inventor: Mayank Goyal, Calgary (CA)

(21) Appl. No.: 19/565,285

(22) Filed: Mar. 12, 2026

Related U.S. Application Data
(60) Provisional application No. 63/770,853, filed on Mar. 12, 2025.
(60) Provisional application No. 63/875,150, filed on Sep. 3, 2025.

(51) Int. Cl. A61B 17/12; A61B 17/00; A61K 31/445; A61L 24/00

(52) U.S. Cl. CPC A61B 17/12186; A61B 17/12109; A61K 31/445; A61L 24/001; A61L 24/0015; A61L 24/0031; A61B 2017/00862; A61B 2017/00893; A61B 2017/1205; A61L 2300/402; A61L 2300/602

(57) ABSTRACT

Pharmaceutical embolizing agents and methods of use thereof to treat pain and other conditions include a medical gel mixed with a radiopaque compound and a sustained release treatment drug compound. The pharmaceutical embolizing agent is injected into a patient through a catheter in order to form an embolus in the patient's vessel, such as the middle meningeal artery, and slowly release the treatment drug over time without being moved or displaced.

### CROSS-REFERENCE

The present application relies on U.S. Patent Provisional Application No. 63/770,853, titled “Sustained Release Liposomal Pharmaceutical Embolizing Agents for Treating Chronic Subdural Hematoma, Associated Headaches and Intractable Migraines” and filed on Mar. 12, 2025, for priority, which is herein incorporated by reference in its entirety.

The present application also relies on U.S. Patent Provisional Application No. 63/875,150, titled “Sustained Release Liposomal Pharmaceutical Embolizing Agents for Treating Chronic Subdural Hematoma, Headaches Including Intractable Migraines, and Other Pain Conditions” and filed on Sep. 3, 2025, for priority, which is herein incorporated by reference in its entirety.

### FIELD

The present specification is related generally to the field of sustained release therapeutic compositions. More specifically, the present specification is related to methods and systems for endovascular intra-arterial delivery of sustained release pharmaceutical embolizing agents which may be used for treating chronic subdural hematoma(s), headaches including intractable migraines, post-craniotomy pain or post-operative pain such as post-operative spine and knee pain.

### BACKGROUND

A chronic subdural hematoma (cSDH) is an organized, encapsulated blood collection in the space between the dura mater and arachnoid mater, i.e. the subdural space. There are many risk factors for the development of cSDH, which include trauma, anticoagulation/antiplatelet use, alcohol consumption, arterial hypertension, cerebrovascular atherosclerosis, diabetes, and brain atrophy. The incidence of cSDH is significantly higher in elderly populations (>70 years) which ranges from 2.0-58 per 100,000. Overall, the cSDH incidence is of 8.2-14.0 cases per 100,000 people annually.

The traditional theory was that most cSDH results from a tear in the bridging veins, which first causes accumulation of an acute SDH that becomes chronic over time. However, this theory has been abandoned in recent years, and it has now been established that the driving factor in the formation of cSDH is not venous injury, but rather injury to the cells lining the inner border of dura mater called the dural border cells. When damaged, the injured cells result in an inflammatory reaction, leading to fibrogenesis and neo-angiogenesis. Various inflammatory mediators are recruited to repair these damaged cells as a part of body's repair mechanism, which results in activation of pro-collagens forming neo-membranes. This in turn leads to the formation of new, fragile capillaries to provide blood supply to these neo-membranes. It is these capillaries that cause repeated slow, insidious bleeding, thereby feeding the hematoma, causing the re-accumulation and expansion of SDH. This leakage perpetuates the inflammatory response and stimulates further membrane development, creating a vicious cycle.

The main arterial supply to the dura mater is through the Middle Meningeal Artery (MMA), which supplies more than two-thirds of the cranial dura mater. MMA is also shown to be the main feeding branch of the newly formed fragile leaky capillaries supplying the neo-membranes. Performing MMA embolization (MMAE), a treatment that is now commonly used to treat cSDH, interrupts blood supply to these neo-membranes and interrupts the inflammatory neoangiogenic cascade, which facilitates hematoma resolution and reduces the risk for hematoma recurrence or progression.

Patients presenting with cSDH often experience headaches due to the pressure exerted by the hematoma on the meninges. MMAE targets the vascular supply to the hematoma membranes, promoting hematoma resolution and subsequently reducing headache symptoms. MMAE has been investigated as a novel therapeutic approach for patients with refractory migraines. The procedure aims to modulate dural blood flow, potentially impacting migraine pathophysiology.

The MMA carries dense perivascular innervation from the ophthalmic branch (V1) of the trigeminal nerve. These perivascular trigeminal afferents are directly implicated in the cortical spreading depression-trigeminovascular activation-meningeal neurogenic inflammation cascade underlying migraine pathophysiology. Embolization of the MMA with the pharmaceutical embolizing agent of the present specification acts through a dual mechanism: (1) mechanical reduction of arterial dilation and pulsatility in meningeal vessels, reducing the mechanical stimulus to perivascular nociceptors; and (2) direct pharmacological blockade and sustained neuroinflammatory modulation of perivascular trigeminal afferents via controlled local release of bupivacaine. This constitutes a mechanistically distinct approach from systemic CGRP antagonists, triptans, or intravenous lidocaine, which lack the spatial specificity and duration of effect achievable through targeted intra-arterial depot deposition.

Currently, cSDH, including associated headaches and intractable migraines, is treated by injecting embolic materials, such as liquid agents, or deploying metal coils into the MMA to obstruct blood flow, thereby reducing arterial dilation and promoting hematoma resolution. Broad categories of liquid embolic agents include super-glue based agents, EVOH (ethylene-vinyl alcohol) based agents such as, for example, Onyx (an ethylene-vinyl alcohol copolymer (EVOH) and dimethyl sulfoxide (DMSO) solution) and PVA (polyvinyl alcohol) particles. Other examples of liquid embolic agents comprise Squid which is a non-adhesive liquid embolic agent, PHIL which is a non-biodegradable iodine based liquid embolic, Lipiodol which is a mixture of iodinated poppyseed oil fatty acids, N-butyl-2-cyanoacrylate which is a clear liquid that polymerizes to form a solid cast, and Silk Elastin Protein Liquid Embolic (SELP) which is a silk elastin hydrogel with low viscosity.

The use of liquid embolizing agents or metal coils may be advantageous in that these techniques are well-established and effective, and physicians are familiar with using them as treatment modalities. There are disadvantages, however, including 1) that the occlusion is permanent (it is possible the permanent devascularization of the dura is not good in the long run, for example, some initial literature suggests that the pulsatility of the MMA is helpful in glymphatic drainage of the brain, 2) if the glue goes in the wrong location it can lead to serious complications (sometimes the glue does not move forward and does not result in deep penetration into the dura), and 3) access to the vessel is permanently lost in case of recurrence of disease.

In embodiments, the non-permanent nature of the pharmaceutical embolizing agent is further advantageous in preserving meningeal lymphatic and glymphatic function. Emerging evidence suggests that arterial pulsatility of the MMA and its branches contributes to perivascular cerebrospinal fluid and interstitial fluid exchange (the glymphatic system), facilitating clearance of metabolic waste products from brain tissue. Permanent devascularization of the MMA, as produced by existing embolic agents such as Onyx and n-BCA, may impair this pulsatility-driven glymphatic flow over the long term. The resorbable pharmaceutical embolizing agent of the present specification permits restoration of arterial pulsatility following recanalization, thereby preserving glymphatic function and avoiding the potential long-term consequences of permanent meningeal devascularization.

With respect to pathophysiology, there may be evidence that there is an increased expression of VEGF (vascular endothelial growth factor) in the dura leading to hypervascularity. In some cases, increased expression of VEGF has been treated using Bevacizumab (anti-VEGF) and other similar drugs. However, the problem is that Bevacizumab passes through the system and does not have enough duration of contact both in terms of dosage and duration and therefore lacks the requisite longevity in the system for efficacy. What is therefore needed is a higher local concentration and greater duration of action.

Intractable headaches are persistent headaches that fail to respond to standard or aggressive treatments. These headaches may include primary types—such as migraine or tension-type headache—and may also result from chronic daily headache disorders, medication overuse, or secondary causes that have been ruled out with imaging and diagnostic testing. The most frequent forms are chronic migraine, tension-type headache, cluster headache, and less common primary headaches such as hemicrania continua and new daily persistent headache. Triggers include stress, changes in sleep routine, light, sound, fast movement, and, for some patients, overuse of acute headache medications leading to medication-overuse headache. Intractable headaches are severely disabling, often affecting the patient's daily life, work, and emotional well-being. There is no cure for intractable headaches, and treatment is focused on reducing frequency and severity. Standard pharmacologic strategies may include beta-blockers, anticonvulsants, antidepressants, and neurotoxins for prevention, but some cases remain resistant to all options. Thus, intractable headaches represent a major clinical challenge, requiring careful diagnosis, individualized therapy, and sometimes advanced or interventional procedures to achieve relief.

Accordingly, there is need for an embolizing agent that is not permanent, that is easier to handle while lowering the risk of complication even if delivered or if it travels to the wrong location, and that “attacks” or treats the actual pathophysiological condition rather than only the symptom that can be used to treat chronic subdural hematoma(s), headaches including intractable migraines, low back pain, and post-craniotomy pain or post-operative pain such as post-operative spine and knee pain.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a method of treating pain in a patient, the method comprising: forming a vascular access into the patient; advancing a catheter through the patient's vasculature to a target artery supplying blood to a pain-associated anatomical region; and administering through the catheter a pharmaceutical embolizing agent to a location in said target artery, wherein the pharmaceutical embolizing agent comprises an injectable biocompatible medical gel and a sustained-release analgesic incorporated within the medical gel, wherein the pharmaceutical embolizing agent is adapted to (i) form an embolus at said location, (ii) release a therapeutic drug at said location over time, and (iii) be structurally stabilized at the location to retain the analgesic without migration within the target artery and release the analgesic over an extended period to reduce pain experienced by the patient.

Optionally, the method further comprises administering the pharmaceutical embolizing agent by injection such that the analgesic is released at least 5 cm beyond the patient's main middle meningeal artery trunk into distal meningeal branches to thereby achieve perivascular contact between the analgesic and trigeminal afferents innervating the patient's distal meningeal territory.

Optionally, the sustained-release analgesic comprises bupivacaine and functional equivalents thereof.

Optionally, the sustained-release analgesic is encapsulated within liposomes, microspheres, or nanoparticles dispersed in the medical gel.

Optionally, the pharmaceutical embolizing agent is configured to fully release all of the analgesic over a period of at least 2 weeks and less than 10 weeks following administration.

Optionally, the medical gel comprises a hydrogel comprising at least one of polyethylene glycol hydrogels, chitosan hydrogels, alginate hydrogels, fibrin hydrogels, hyaluronic acid hydrogels, and PLGA-based gels.

Optionally, the pharmaceutical embolizing agent exhibits shear-thinning rheological characteristics enabling delivery through a microcatheter and structural recovery after deposition within the target artery.

Optionally, the pharmaceutical embolizing agent has a viscosity between approximately 50 and 500 centipoise during injection.

Optionally, the pharmaceutical embolizing agent is biodegradable and non-permanent, allowing recanalization of the target artery over time.

Optionally, the pharmaceutical embolizing agent further comprises a radiopaque material enabling visualization under fluoroscopic imaging. Optionally, the radiopaque material comprises at least one of tantalum, tungsten, barium, and polymer-bound iodine.

Optionally, administering the pharmaceutical embolizing agent comprises injecting the agent through a microcatheter having an inner diameter between approximately 0.0135 and 0.0314 inches.

Optionally, administering the pharmaceutical embolizing agent comprises injecting the pharmaceutical embolizing agent at a rate between approximately 0.01 mL/s and 0.05 mL/s. Optionally, the target artery comprises a middle meningeal artery.

Optionally, the pain comprises at least one of headache pain, migraine pain, post-craniotomy pain, post-operative pain, spine pain, knee pain, and low back pain associated with spinal structures supplied by lumbar arterial branches.

Optionally, an amount of the analgesic is less than approximately 10% by weight of the pharmaceutical embolizing agent.

Optionally, the location is defined by a region extending 0.1 cm to 25 cm into the target artery from an injection site.

Optionally, after said administration, the patient experiences a reduction of at least 30% in a pain score measured using a standardized pain assessment scale.

Optionally, the pharmaceutical embolizing agent exhibits at least one of a power-law flow behavior index between approximately 0.3 and 0.4 and a viscosity between 50 and 500 cP at shear rates of 0.1-10 s⁻¹.

Optionally, once administered at the location, the medical gel has an elastic modulus (G′) of 10 to 500 Pascals, such that the administered medical gel deforms compatibly with physiological arterial wall pulsation.

In some other embodiments, the present specification describes a method of treating pain in a patient, the method comprising: forming a vascular access into the patient; advancing a microcatheter having an inner diameter between approximately 0.0135 and 0.027 inches through the patient's vasculature to a target artery supplying a pain-associated anatomical region; and injecting a pharmaceutical embolizing agent through the catheter wherein the pharmaceutical embolizing agent comprises an injectable biocompatible medical gel and a sustained-release analgesic incorporated within the medical gel, wherein the pharmaceutical embolizing agent is injected at a rate between approximately 0.01 mL/s and 0.05 mL/s and wherein the pharmaceutical embolizing agent is adapted to (i) mechanically embolize a target vascular region, (ii) release a therapeutic drug locally over time, and (iii) structurally stabilize within the target artery to retain the analgesic at a treatment site without migration within the target artery and release substantially all of the analgesic over a period of 2 to 6 weeks to reduce pain experienced by the patient.

Thus, the pharmaceutical embolizing agent is adapted to be structurally stabilized at the treatment site to retain the analgesic without migration within the target artery and release the analgesic over an extended period to reduce pain experienced by the patient. In embodiments, the treatment site is 0.1 cm to 25 cm, and every numerical increment therein, into the target artery from an injection site.

In some embodiments, the pharmaceutical embolizing agent exhibits shear-thinning rheological characteristics enabling delivery through a microcatheter and structural recovery after deposition within the target artery.

In some embodiments, the present specification describes a pharmaceutical embolizing agent, wherein the first composition further comprises a second therapeutic agent selected from corticosteroids, preferably dexamethasone, co-encapsulated within the medical gel at a dose of 0.5 to 4 mg per administration, providing combined anti-neovascular and anti-inflammatory activity for treatment of chronic subdural hematoma.

In some embodiments, the yield stress of 5 Pa to 20 Pa of the pharmaceutical embolizing agent prevents displacement of the deployed agent by physiological arterial perfusion pressure following cessation of injection, thereby preventing non-target embolization without operator intervention.

In some embodiments, the slow-release anti-VEGF compound is selected from the group consisting of bevacizumab, ranibizumab, aflibercept, and biosimilars or functional equivalents thereof.

In some embodiments, the target artery comprises at least one of the anterior circumflex humeral artery, thoracoacromial artery, or posterior circumflex humeral artery, and the pain condition comprises rotator cuff tendinopathy, subacromial impingement syndrome, or calcific tendinitis of the shoulder.

In some embodiments, the target artery comprises at least one of the radial recurrent artery or posterior interosseous recurrent artery, and the pain condition comprises lateral epicondylitis or refractory elbow tendinopathy.

In some embodiments, the target artery comprises at least one of the medial circumflex femoral artery, lateral circumflex femoral artery, or inferior gluteal artery, and the pain condition comprises chronic hip pain or hip osteoarthritis.

In some embodiments, the target artery comprises at least one of the medial plantar artery, lateral plantar artery, or calcaneal branches of the posterior tibial artery, and the pain condition comprises plantar fasciitis or chronic heel pain.

In some embodiments, the target artery comprises the basivertebral artery or posterior vertebral body branches supplying the basivertebral nerve, and the pain condition comprises discogenic low back pain or vertebrogenic pain associated with vertebral endplate pathology, the embolization providing at least one of: ischemic interruption of basivertebral nerve perfusion; or sustained local delivery of a long-acting local anesthetic to basivertebral nerve terminals.

In some embodiments, the target artery comprises medial branch arteries arising from dorsal branches of lumbar or cervical segmental arteries supplying the zygapophyseal joints, and the pain condition comprises facet-mediated spinal pain, the embolization delivering sustained perivascular local anesthetic to medial branch nerve terminals for at least 2 weeks.

In some embodiments, the target artery comprises the sphenopalatine artery or pterygopalatine branches of the internal maxillary artery, and the pain condition comprises cluster headache, refractory trigeminal neuralgia, or other craniofacial pain syndrome mediated by sphenopalatine ganglion activation.

In some embodiments, the target artery comprises a branch of the occipital artery supplying the greater or lesser occipital nerve territory, and the pain condition comprises occipital neuralgia.

In some embodiments, an optional repeat treatment of a condition in a patient who has previously undergone middle meningeal artery embolization with a pharmaceutical embolizing agent is performed, the method comprising: confirming recanalization of the previously embolized middle meningeal artery by imaging; and administering a second pharmaceutical embolizing agent through a microcatheter to the recanalized middle meningeal artery. In embodiments, the repeat treatment is based on a patient's symptoms and/or clinical judgment.

Optionally, an interval between a first administration and a second administration is at least 4 weeks. Optionally, an interval between a first administration and a second administration is between 4 to 24 weeks.

Optionally, the middle meningeal artery embolization of a patient following administration of a pharmaceutical embolizing agent comprising a hyaluronic acid-based medical gel is reversed, wherein the method comprises administering hyaluronidase intra-arterially to the embolized vessel at a dose sufficient to enzymatically degrade the hyaluronic acid gel matrix and thereby restore blood flow through the target artery.

In some embodiments, the present specification describes a kit for preparing a pharmaceutical embolizing agent, comprising: (a) a first component comprising a gel-forming polymer composition incorporating a sustained-release therapeutic agent encapsulated in liposomes, microspheres, or nanoparticles; and (b) a second component comprising a radiopaque powder, wherein the first and second components are stored separately and combined immediately prior to intra-arterial administration.

Optionally, the radiopaque powder comprises tantalum particles.

Optionally, the kit further comprises a microcatheter sized for intra-arterial delivery.

Optionally, the kit further comprises a vial of hyaluronidase for emergency reversal of embolization.

In some embodiments, the present specification discloses a pharmaceutical embolizing agent comprising a medical gel and a slow-release anti-VEGF compound.

Optionally, the slow-release anti-VEGF compound is Bevacizumab. In embodiments, the anti-VEGF compound is selected from the group consisting of bevacizumab, ranibizumab, aflibercept, and biosimilars or functional equivalents thereof, or any other agent that inhibits vascular endothelial growth factor signalling. Bevacizumab is a preferred embodiment due to its established clinical use, large molecular weight (approximately 149 kDa) which slows diffusion through the gel matrix and supports sustained local retention, and its well-characterised anti-neovascular activity relevant to the pathological neoangiogenesis of subdural neo-membranes in cSDH.

Optionally, the medical gel is selected from the group consisting of polyethylene glycol (PEG)-based hydrogels, chitosan-based hydrogels, fibrin-based hydrogels, alginate-based hydrogels, hyaluronic acid (HA) hydrogels, and poly(lactic-co-glycolic acid) (PLGA) microsphere gels.

Optionally, the agent is formulated for sustained release over a period of 2 to 6 weeks after administration.

Optionally, the agent is radio-opaque. Optionally, radio-opacity is achieved by inclusion of at least one component selected from the group consisting of tantalum powder, tungsten, barium, and polymer-bound iodine.

Optionally, the agent is used to treat chronic subdural hematoma (cSDH).

Optionally, the agent is packaged or encapsulated for controlled, targeted delivery within an arterial vessel at the treatment site.

Optionally, the agent is configured to provide a localized therapeutic concentration at the site of administration, minimizing systemic exposure.

Optionally, the agent has a viscosity in the range of 50 to 500 centipoise at the time of injection. Optionally, the viscosity is optimized such that the embolizing agent exhibits shear-thinning properties to allow smooth delivery through a microcatheter but forms a stable depot at the target site. Optionally, the viscosity allows for manual injection at a rate of 0.01 to 0.05 mL per second through a microcatheter under injection pressure not exceeding 300 mmHg and not exceeding the mean arterial perfusion pressure of the target vessel, whichever is lower, to avoid vessel barotrauma or retrograde flow into non-target territories.

In some other embodiments, the present specification also discloses a pharmaceutical embolizing agent comprising a medical gel and a slow-release analgesic compound.

Optionally, the slow-release analgesic compound is bupivacaine.

Optionally, the medical gel is selected from the group consisting of PEG-based hydrogels, chitosan-based hydrogels, PLGA-based gels, and HA-based gels.

Optionally, the slow-release analgesic compound is encapsulated in at least one carrier selected from liposomes, microspheres, or nanoparticles for controlled, targeted delivery at a treatment site.

Optionally, the agent is radio-opaque. Optionally, radio-opacity is achieved by inclusion of at least one component selected from the group consisting of tantalum powder, tungsten, barium, and polymer-bound iodine.

Optionally, the agent is used to treat a pain condition selected from the group consisting of headaches, intractable migraines, low back pain, post-craniotomy pain, and post-operative pain such as post-operative spine and knee pain.

Optionally, the agent is configured to provide a localized therapeutic concentration at a treatment site of administration, minimizing systemic exposure.

Optionally, the agent has a viscosity in the range of 50 to 500 centipoise at the time of injection. Optionally, the viscosity is optimized such that the embolizing agent exhibits shear-thinning properties to allow smooth delivery through a microcatheter but forms a stable depot at the target site. Optionally, the viscosity allows for manual injection at a rate of 0.01 to 0.05 mL per second through a microcatheter under pressure not exceeding 300 mmHg and not exceeding the mean arterial perfusion pressure of the target vessel, whichever is lower, to avoid vessel barotrauma or retrograde flow into non-target territories.

The present specification also discloses a method of treating a condition selected from chronic subdural hematoma, headache, intractable migraine, low back pain, post-craniotomy pain, or post-operative pain (such as post-operative spine and knee pain) in a patient, the method comprising: forming a puncture into an artery in the patient; inserting one or more co-axial catheters through the puncture and navigating through the patient's blood vessels under X-ray guidance to the target artery; administering, through the catheter, one of the above described pharmaceutical embolizing agents; removing the catheter; and closing the puncture.

Optionally, the pharmaceutical embolizing agent has a viscosity ranging from 50 to 500 centipoise during injection.

Optionally, the pharmaceutical embolizing agent is non-permanent and biodegradable, allowing vessel recanalization over time.

Optionally, the pharmaceutical embolizing agent is administered via intra-arterial injection into the middle meningeal artery (MMA).

Optionally, the pharmaceutical embolizing agent is administered into a genicular artery for treatment of knee pain.

The present specification also discloses a pharmaceutical embolizing agent comprising: a medical gel; and slow-release bevacizumab.

Optionally, said agent is radio-opaque.

Optionally, said agent is used to treat at least one condition of chronic subdural hematomas (cSDH).

The present specification also discloses a pharmaceutical embolizing agent comprising: a medical gel; and a slow-release analgesic.

Optionally, said agent is radio-opaque.

Optionally, said agent is used to treat at least one condition of headaches or intractable migraines.

The present specification also discloses a method of administering a pharmaceutical embolizing agent, comprising: forming a puncture into an artery in at least one of a groin or wrist of a patient; inserting a series of co-axial catheters through the incision and threading the catheters through the patient's blood vessels, under X-ray guidance, until the catheters reach the middle meningeal artery of the patient; administering, through the catheters, the pharmaceutical embolizing agent; removing the catheters; and closing the incision.

Optionally, the pharmaceutical embolizing agent is at least one of a first composition or a second composition, wherein the first composition comprises a medical gel mixed with slow-release bevacizumab and wherein the second composition comprises a medical gel mixed with a slow-release analgesic.

Optionally, the pharmaceutical embolizing agent has a viscosity ranging from 50 to 500 centipoise during injection.

Optionally, the pharmaceutical embolizing agent has a prolonged release of 2 to 6 weeks after administering.

Optionally, the pharmaceutical embolizing agent includes a medical gel that is non-permanent so that the middle meningeal artery recanalizes over a period of time.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A is a flowchart describing exemplary steps for preparation and administration of a pharmaceutical embolizing agent, in accordance with some embodiments of the present specification;

FIG. 1B is a flowchart describing exemplary steps of a method of administering a pharmaceutical embolizing agent to treat a patient's condition, in accordance with some embodiments of the present specification;

FIG. 2 is a flowchart describing exemplary steps of a method of treating cSDH, in accordance with some embodiments of the present specification;

FIG. 3 is a flowchart describing exemplary steps of a method of treating headaches, intractable migraine, and post-craniotomy pain conditions, in accordance with some embodiments of the present specification;

FIG. 4 is a flowchart describing exemplary steps of a method of treating post-operative pain spine and knee pain, in accordance with some embodiments of the present specification;

FIG. 5 is a flowchart describing exemplary steps of a method of treating low back pain, in accordance with some embodiments of the present specification;

FIG. 6 is a flowchart describing exemplary steps of a method for treating shoulder pain and rotator cuff tendinopathy, in accordance with some embodiments of the present specification;

FIG. 7 is a flowchart describing exemplary steps of a method for treating lateral epicondylitis (i.e. tennis elbow) and elbow tendinopathy, in accordance with some embodiments of the present specification;

FIG. 8 is a flowchart describing exemplary steps of a method for treating chronic hip pain and hip osteoarthritis, in accordance with some embodiments of the present specification;

FIG. 9 is a flowchart describing exemplary steps of a method for treating plantar fasciitis and heel pain, in accordance with some embodiments of the present specification;

FIG. 10 is a flowchart describing exemplary steps of a method for treating discogenic low back pain, in accordance with some embodiments of the present specification;

FIG. 11 is a flowchart describing exemplary steps of a method for treating facet-mediated pain, in accordance with some embodiments of the present specification;

FIG. 12 is a flowchart describing exemplary steps of a method for treating craniofacial pain, in accordance with some embodiments of the present specification;

FIG. 13 is a flowchart describing exemplary steps of a method for treating occipital neuralgia, in accordance with some embodiments of the present specification;

FIG. 14 is a diagrammatic illustration of a rheological profile of the pharmaceutical embolizing agent during the stages of injection and deployment within a blood vessel, in accordance with some embodiments of the present specification; and

FIG. 15 illustrates a double sustained-release architecture/system for delivering a therapeutic agent using a two-tier drug delivery system, in accordance with some embodiments of the present specification.

### DETAILED DESCRIPTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

The term VEGF, used in the disclosure, may refer to a vascular endothelial growth factor which is a signal protein produced by cells that stimulates the formation of blood vessels. Specifically, VEGF is a sub-family of growth factors, the platelet-derived growth factor family of cystine-knot growth factors. They are signaling proteins involved in both vasculogenesis and angiogenesis.

The term “cP”, used in the disclosure, refers to ‘centipoise’ which is a unit of viscosity in the CGS system.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

The present specification is directed towards a pharmaceutical embolizing agent that is used in conjunction with a system for administering the pharmaceutical embolizing agent to a patient for treating chronic subdural hematomas (cSDH), headaches, intractable migraines and post-craniotomy pain or post-op pain through MMAE (middle meningeal artery embolization). In other embodiments, the pharmaceutical embolizing agents and methods and systems of administration, are used to treat post-operative spine pain and knee pain.

In one embodiment, treatment achieves a clinically meaningful reduction in patient pain scores, defined as a decrease of at least 30% on the Visual Analog Scale (VAS) or Numeric Rating Scale (NRS) from baseline, representing the minimum clinically important difference (MCID) for headache and post-operative pain. In embodiments directed to cSDH treatment, the cSDH recurrence rate following middle meningeal artery embolization is less than 5%. Thus, a reduction in pain is associated and/or correlated with an overall decrease in the occurrence of conditions including chronic migraine, cluster headache, and post-craniotomy or post-op pain.

In various embodiments, patient pain, including pain associated with cSDH, chronic/intractable migraine, cluster headache, post-craniotomy or post-op conditions such as post-operative spine and knee pain, low back pain, and other pain-related disorders, may be assessed using standardized and validated pain assessment tools to quantify both severity and functional impact. In various embodiments, patient pain is evaluated using one or more of the following instruments: the Visual Analog Scale (VAS), the Numeric Rating Scale (NRS), the Verbal Rating Scale (VRS), the McGill Pain Questionnaire (MPQ), and the Brief Pain Inventory (BPI).

The Visual Analog Scale (VAS) utilizes a linear continuum on which the patient marks pain intensity between “no pain” and “worst imaginable pain,” thereby providing a sensitive and continuous measure of pain change. The Numeric Rating Scale (NRS) employs a numerical scale, typically ranging from 0-10 or 0-100, allowing patients to quantify pain severity in a reproducible and easily interpretable manner. The Verbal Rating Scale (VRS) uses categorical descriptors such as mild, moderate, or severe to facilitate straightforward classification of pain intensity. The McGill Pain Questionnaire (MPQ) provides a multidimensional assessment by capturing both qualitative and quantitative characteristics of pain through structured descriptors. The Brief Pain Inventory (BPI) evaluates both pain severity and the degree to which pain interferes with daily activities, thereby reflecting functional outcomes.

For headache-related disorders, including chronic migraine and cluster headache, headache-specific assessment tools may additionally be employed in conjunction with the general pain scales described above. These include the Headache Impact Test (HIT-6), which quantifies the effect of headache on quality of life, and the Migraine Disability Assessment Scale (MIDAS), which evaluates headache-related impairment in work, household, and social activities. These instruments help quantify headache-related disability, overall migraine severity, and impact on daily life.

Cluster headaches may be diagnosed primarily through clinical evaluation, with attention to characteristic pain patterns, anatomical location, duration of episodes, and accompanying autonomic symptoms. In some embodiments, physical and neurological examinations are conducted to exclude secondary causes of headache, and neuroimaging modalities such as magnetic resonance imaging (MRI) or computed tomography (CT) may be utilized when alternative etiologies are suspected.

For post-craniotomy pain and other post-operative pain conditions, standardized assessment tools such as the Numeric Rating Scale (NRS) and Visual Analog Scale (VAS), described above, may be specifically employed to quantify acute or subacute pain intensity. In certain cases, observational assessments of acute pain behavior may be used, particularly for patients who are unable to verbally communicate their pain experience.

For post-operative spine and knee-related pain, both subjective and objective measures may be used, optionally in combination with the general pain scales described herein. Subjective assessments include instruments such as the Visual Analog Scale (VAS), the Numeric Rating Scale (NRS), the Oswestry Disability Index (ODI) for spine-related functional impairment, and the Knee Injury and Osteoarthritis Outcome Score (KOOS), which evaluates knee pain, symptoms, and functional limitations following surgical intervention. Objective measures may include functional performance tests such as the 30-second chair stand test and the 40-meter fast-paced walk test, which provide quantifiable indicators of physical impairment and treatment response.

Accordingly, pain assessment in the context of the present specification may involve a combination of quantitative intensity scales, multidimensional questionnaires, headache-specific disability tools, functional outcome measures, and clinical examinations, thereby enabling comprehensive evaluation of therapeutic efficacy across multiple pain-related conditions.

Using the embodiments of the present specification, the average decrease in pain scores in all of the aforementioned conditions is at least 30% of the pre-treatment pain score.

### Pharmaceutical Embolizing Agent—Overview and Characteristics

In embodiments, the pharmaceutical embolizing agent described in the present specification comprises a gel-based formulation including an injectable medical-grade drug-delivery gel (hereinafter also referred to as a “medical gel” or “gel”) in combination with a slow- or sustained-release therapeutic agent, medicament, or drug. The therapeutic agent may include, for example, an anti-vascular endothelial growth factor (anti-VEGF) compound such as, but not limited to, bevacizumab in a first composition, or an analgesic compound such as bupivacaine in a second composition. The gel serves as a biocompatible delivery matrix configured to facilitate localized embolization and controlled release of the incorporated therapeutic agent at a target vascular site.

It should be noted herein that the term “pharmaceutical embolizing agent” as used in the discussion of the present section refers to both the first composition and the second composition. Separate discussions are provided for the embodiments in which the first composition differs from the second composition.

In some embodiments, the pharmaceutical embolizing agent is specifically configured for targeted retention within a selected vascular territory, such as the middle meningeal artery (MMA). The pharmaceutical embolizing agent is configured to remain localized within the intended anatomical region or location in the target artery without clinically significant migration while also being capable of delivery via a microcatheter.

Thus, in embodiments, the present specification describes the administration of a pharmaceutical embolizing agent, through a catheter, to a location in a target artery. In embodiments, the pharmaceutical embolizing agent is adapted to form an embolus at the location and release a therapeutic drug at the location over time. In addition, the pharmaceutical embolizing agent is adapted to be structurally stabilized at the location to retain the therapeutic drug without migration within the target artery and release the therapeutic drug over an extended period of time to reduce pain experienced by the patient. In embodiments, the location is defined by a region that has a size that can extend 0.1 cm to 25 cm, and every numerical increment therein, from the injection site within the target artery.

In some embodiments, the extended period of time is dependent upon the formulation of the pharmaceutical embolizing agent. In some other embodiments, the extended period of time is dependent upon the formulation of the therapeutic drug. In some other embodiments, the extended period of time can be manipulated based on the chemical formulation of the pharmaceutical embolizing agent and/or therapeutic drug. In some other embodiments, the extended period of time is at least 1 day. In some other embodiments, the extended period of time ranges from 1 day to 8 weeks, or any numerical increment therein. In some other embodiments, the extended period of time ranges from 2 weeks to 6 weeks. In some other embodiments, the extended period of time ranges from 1 day to 8 weeks. In some other embodiments, the extended period of time supports a double sustained-release formulation, having a first time period and a second time period. In some embodiments, the first time period is on the order of at least 1 day. In some embodiments, the first time period is on the order of 72 hours. In some embodiments, the second time period is on the order of at least an additional 2 weeks to 6 weeks. In some embodiments, the first time period support liposomal-based release. In some embodiments, the second time period supports hydrogel-based sustained release. In some embodiments, the first time period overlaps with the second time period. In some embodiments, the first time period is encompassed by the second time period.

The pharmaceutical embolizing agent is further characterized by a plurality of advantageous properties, including, but not limited to, transient or non-permanent behavior within the vasculature, such that the MMA is permitted to recanalize over a period of time following treatment, ease of handling and delivery, reduced risk of procedure-related complications, therapeutic action directed toward an underlying pathophysiological condition, and radiopacity sufficient to permit visualization under imaging guidance.

To achieve targeted retention and deliverability within the MMA, the pharmaceutical embolizing agent exhibits a controlled balance between viscosity and flowability. The viscosity is sufficient to enable the pharmaceutical embolizing agent to remain in place following deposition within the MMA, while the flowability permits advancement of the pharmaceutical embolizing agent through a microcatheter lumen during injection.

### Rheological Characteristics

In accordance with some aspects of the present specification, this balance between viscosity and flowability is achieved, at least in part, through shear-thinning rheological characteristics (i.e. viscosity, elasticity, and shear response). Preferably, the pharmaceutical embolizing agent maintains a relatively high viscosity under low-shear conditions, thereby resisting unintended migration, and exhibits reduced viscosity when pressure or shear is applied, thereby enabling flow through a thinner lumen, such as that of a microcatheter. Stated differently, the shear-thinning rheological characteristics enable the pharmaceutical embolizing agent to flow under catheter-relevant shear conditions and structurally recover upon deposition within a target vessel, such as the middle meningeal artery.

In some embodiments, during injection, the pharmaceutical embolizing agent has a viscosity between approximately 50 and 500 centipoise at low to moderate shear rates (such as, for example, at shear rates of approximately 0.1-10 s⁻¹), a power-law flow behavior index (n) between approximately 0.3 and 0.4, and a consistency index (K) between approximately 100 and 300 Pa·sⁿ, where n is the power-law flow behavior index.

The pharmaceutical embolizing agent further exhibits a yield stress ranging from approximately 5 to 20 Pascals, which may be characterized as sufficiently low to prevent premature backflow while remaining adequate to resist reflux once deployed. The yield stress may be adjusted, in embodiments, through the use of low concentrations of physical crosslinkers or polymer entanglements. Importantly, the yield stress of 5 to 20 Pascals exceeds the wall shear stress generated by physiological arterial perfusion pressure in the MMA (estimated at approximately 1 to 3 Pascals in small meningeal branches), such that the deployed gel resists displacement by normal blood flow following cessation of injection. This property functions as a built-in flow-stop mechanism: once injection pressure is released, the gel immediately re-gels at the deposition site and is not carried distally by arterial flow into non-target capillary beds or venous circulation. This self-arresting behavior is a critical safety feature that distinguishes the pharmaceutical embolizing agent from liquid embolic agents lacking yield stress, which continue to flow after injection ceases and require precise operator timing to prevent non-target embolization.

Under catheter-relevant shear rates (approximately 10⁴-10⁵ s⁻¹), the pharmaceutical embolizing agent exhibits shear-thinned viscosity values of less than approximately 10 cP, consistent with its power-law rheological behavior. Viscosity, as a measure of resistance to flow, decreases as shear rate increases, thereby enabling delivery through a microcatheter, including a 0.017-inch microcatheter, at flow rates of 0.01-0.05 mL/s under pressures below 300 mmHg and not exceeding the mean arterial perfusion pressure of the target vessel, whichever is lower, to avoid vessel barotrauma or retrograde flow into non-target territories. It should be noted that the mean arterial perfusion pressure varies with the type of target vessel as is known to those of skill in the art.

The shear-thinning behavior of the pharmaceutical embolizing agent further minimizes mechanical injury to the vessel wall during injection. At the catheter tip, the gel undergoes a rapid transition from high-shear (approximately 10⁴-10⁵ s⁻¹) to near-zero shear as it exits into the vessel lumen. This transition involves extensional flow at the catheter outlet, which is managed by the gel's viscoelastic properties: the elastic component (G′) of the gel absorbs and dissipates extensional stress, preventing the concentrated pressure from being transmitted directly to the vessel wall. In embodiments, the pharmaceutical embolizing agent exhibits thixotropic behavior, wherein the gel structure, once disrupted by shear during catheter transit, recovers progressively over a period of seconds to minutes following deposition. This controlled recovery window allows the gel to conform to vessel geometry before solidifying, promoting uniform contact with the vessel wall without mechanical stress concentration. The elastic modulus (G′) of the pharmaceutical embolizing agent in its deployed state is preferably in the range of 10 to 500 Pascals, a range compatible with the elastic modulus of the MMA wall (estimated 10 to 100 kPa at physiological pressures). Thus, the gel is formulated such that it deforms compatibly with arterial wall pulsation rather than creating stress concentrations at the gel-vessel interface that could cause endothelial injury or vessel rupture over time.

In embodiments, the shear-thinning rheological characteristics of the medical gel constituting the pharmaceutical embolizing agent are maintained even when a medicament, such as bevacizumab or bupivacaine, is incorporated therein.

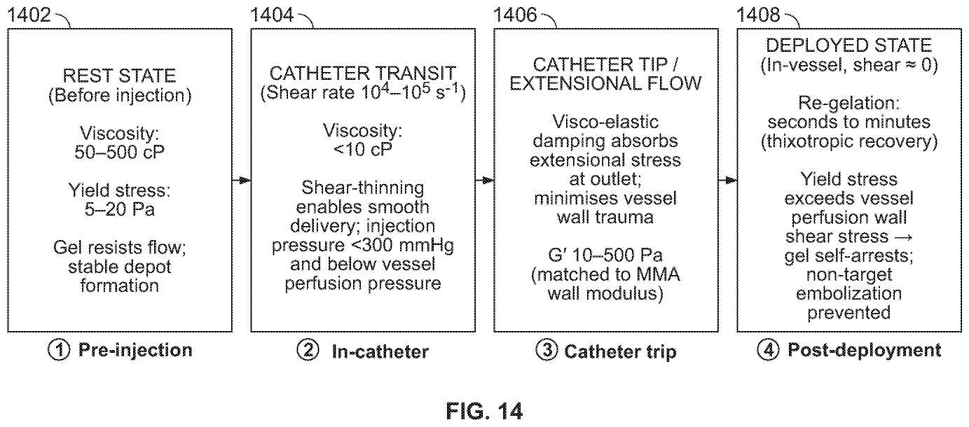

FIG. 14 is a diagrammatic illustration of a rheological profile of the pharmaceutical embolizing agent during stages of injection and deployment within a blood vessel, in accordance with some embodiments of the present specification. FIG. 14 presents the mechanical and flow characteristics of the embolizing agent across four sequential stages, represented by separate boxes corresponding to a rest state (pre-injection), catheter transit, catheter tip flow, and deployed state (post-deployment). Together, these stages demonstrate how the embolizing agent transitions between low-viscosity injectable behavior and a stabilized in-vessel gel that resists downstream migration.

Referring to the first box 1402, in the rest state or before injection (pre-injection condition), the pharmaceutical embolizing agent or gel exhibits a viscosity in the range of approximately 50-500 centipoise (cP) and a yield stress of approximately 5-20 Pa. In this state, the gel resists spontaneous flow and maintains structural integrity, thereby supporting stable depot formation once deployed in the vasculature. The yield stress prevents undesired movement of the gel when it is not subjected to an applied injection force.

Referring to the second box 1404, during catheter transit, the pharmaceutical embolizing agent experiences high shear conditions (shear rate 10⁴-10⁵ s⁻¹) while moving through a microcatheter lumen. Under these shear conditions, the material exhibits shear-thinning behavior, causing its effective viscosity to decrease to less than approximately 10 cP. This reduction in viscosity allows smooth delivery through the catheter while maintaining controlled injection pressures, typically below approximately 300 mmHg, and remaining below physiological perfusion pressures of the target vessel.

Referring to the third box 1406, as the embolizing agent exits the catheter tip and enters the lumen of the target vessel, it undergoes extensional flow. In this region, the material exhibits viscoelastic damping, which absorbs extensional stresses at the outlet of the catheter. This property minimizes trauma to the vessel wall during injection. In some embodiments, the gel may exhibit a storage modulus (G′) of approximately 10 Pa to 500 Pa, which is selected to approximate or match the mechanical modulus of the middle meningeal artery (MMA) wall, thereby improving compatibility between the material and the surrounding vascular environment.

Referring to the fourth box 1408 (deployed state: in-vessel, shear≈0), once injection ceases, the embolizing agent is in a deployed state (or already within the vessel, in which the shear is approximately 0) and rapidly undergoes re-gelation, typically within seconds to minutes due to thixotropic recovery. In this deployed state, the embolizing agent's yield stress again becomes dominant and exceeds the local vessel wall perfusion shear stress, causing the gel to self-arrest within the vessel lumen. This self-arresting behavior stabilizes the embolic depot and prevents distal migration, thereby reducing the likelihood of non-target embolization.

The embolizing agent has several critical design characteristics. Specifically, the yield stress of approximately 5 Pa to 20 Pa exceeds the physiological MMA wall shear stress of approximately 1 Pa to 3 Pa. As a result, after injection ceases and the gel re-solidifies, the embolizing agent resists displacement by arterial blood flow. This self-arresting mechanism enables the material to remain localized without requiring additional operator intervention. It should be noted that comparable self-arresting rheological behavior is generally not present in conventional liquid embolic agents such as such as an ethylene-vinyl alcohol copolymer dissolved in an organic solvent, a cyanoacrylate-based adhesive, or a precipitating hydrophobic injectable liquid composition.

### Encapsulation of the Therapeutic Agent

In some embodiments, the therapeutic agent is incorporated into the medical gel in an encapsulated form to provide controlled and sustained local release. In some embodiments, the therapeutic agent is packaged within liposomes, microspheres, or nanoparticles for slow release at the site of embolization. The contribution of the medicament to overall viscosity, yield stress, and flow behavior is accounted for in the formulation, along with administration of an appropriate therapeutic dosage.

The addition of an encapsulated drug, including liposome-, microsphere-, or nanoparticle-based systems, increases the solid fraction of the pharmaceutical embolizing agent, which in turn can raise viscosity, especially under low-shear conditions. Since viscosity decreases as shear rate increases in shear-thinning systems, such increases are typically more pronounced at or near zero-shear conditions. The resulting increase in structural organization within the gel may also elevate yield stress.

For example, incorporation of approximately 5-10% polylactic-co-glycolic aci) (PLGA) microspheres may increase zero-shear viscosity by approximately 50-200%. Similarly, addition of a slow-release formulation of a therapeutic agent (such as, bevacizumab or bupivacaine) to a shear-thinning medical gel may increase low-shear viscosity, potentially raising yield stress and affecting injectability.

In embodiments in which therapeutic agent-loaded liposomes, microspheres, or nanoparticles are dispersed within the gel matrix, the surrounding gel may prevent post-injection migration of the microspheres and significantly hinder diffusion of the therapeutic agent away from the target site. The gel matrix may therefore enhance localization of the therapeutic agent, resulting in higher local drug concentrations and reduced systemic exposure or side effects.

The degree of impact on injectability depends on multiple factors, including concentration of the medicament, its physical form (e.g., encapsulated versus dissolved), and its physicochemical interactions with the gel matrix. In embodiments, injectability may be preserved by compensatory formulation adjustments, such as employing a more strongly shear-thinning base gel or reducing overall polymer concentration. Any formulation changes may be validated through characterization of a complete rheological profile to confirm that desired injection performance and embolic behavior are maintained.

Suitable lipid-based sustained-release drug delivery technologies use multivesicular liposomes (MVLs), which comprise large, honeycomb-like lipid particles having a median diameter of approximately 24-31 μm. These MVLs contain multiple non-concentric aqueous chambers separated by lipid membranes, enabling gradual erosion or reorganization of the lipid structure and sustained release of the encapsulated drug at the local injection site. Such systems are designed for localized delivery and are not intended for intravenous administration.

In other embodiments, the pharmaceutical embolizing agent comprises well-dispersed and stable liposomes having a particle size of less than or equal to 100 nm, thereby maintaining uniform distribution within the gel matrix and minimizing impact on rheological performance.

In some embodiments, the pharmaceutical embolizing agent comprising a medical gel and liposomal bevacizumab is formulated to provide an extended effect of double-sustained release since the liposomes release over 72 hours and the hydrogel prolongs the release to an extended time period ranging from 2 to 6 weeks.

In further embodiments, the first composition, that already includes a first therapeutic agent (that is, an anti-VEGF such as bevacizumab), further comprises a second therapeutic agent co-encapsulated within the medical gel, providing a combination pharmacological approach to cSDH treatment. In a preferred embodiment, the second therapeutic agent is a corticosteroid, preferably dexamethasone, encapsulated in liposomes, microspheres, or nanoparticles within the gel matrix. The rationale for this combination is that cSDH pathophysiology involves at least two distinct and therapeutically addressable processes: (a) pathological neoangiogenesis of the outer subdural membrane, addressable by the anti-VEGF agent; and (b) neuroinflammation and membrane permeability driven by inflammatory mediators including prostaglandins and cytokines, addressable by local corticosteroid delivery. Dexamethasone is already used systemically for cSDH management; the present embodiment provides targeted local delivery at substantially lower systemic doses, reducing the systemic side-effect profile of prolonged corticosteroid use. The dexamethasone dose in such embodiments ranges from approximately 0.5 to 4 mg per administration, encapsulated to provide sustained release over 2 to 6 weeks consistent with the gel matrix degradation timeline. In embodiments, the combination pharmacological approach provides combined anti-neovascular and anti-inflammatory activity for treatment of chronic subdural hematoma.

In some embodiments, the pharmaceutical embolizing agent comprising a medical gel and liposomal bupivacaine is formulated to provide an extended effect of double-sustained release since the liposomes release over 72 hours and the hydrogel prolongs the release to an extended time period ranging from 2 to 6 weeks.

### Radiopacity

In embodiments, the pharmaceutical embolizing agent is configured to have substantially uniform radiopacity throughout its volume in order to guide and confirm arterial delivery under imaging, such as fluoroscopy-guided injection. The embolizing agent is therefore compatible with fluoroscopic visualization and is formulated to prevent sedimentation of radiopaque constituents, thereby enabling real-time imaging guidance during embolization.

In various embodiments, radiopacity may be achieved through incorporation of one or more radiopaque materials, including, for example, tungsten, barium, polymer-bound iodine, or tantalum.

In some embodiments, tantalum powder is used as a radiopaque agent due to its high atomic number and chemical inertness. When incorporated into a polymer matrix, tantalum enables the injected material to be visualized under X-ray imaging.

In embodiments utilizing tantalum, a stable and uniform dispersion of the radiopaque material within the gel is maintained to ensure homogeneous radiopacity. For example, tantalum may be provided in a fine, micronized form, such as particles having a size range of approximately 10-50 μm, and incorporated at concentrations of approximately 35% w/v. The powder is evenly distributed throughout the gel to ensure uniform visibility under fluoroscopy, as any settling may result in inhomogeneous radiopacity. Uniform dispersion may be achieved through thorough mixing techniques, including vigorous shaking or inversion.

In order to mitigate sedimentation and enhance shelf-life stability, the pharmaceutical embolizing agent may be engineered to exhibit increased viscosity during storage to slow particle movement, while permitting reduced viscosity at the time of injection. In some embodiments, viscosity reduction at the time of injection may be achieved through warming or dilution. Since low viscosity is desirable for injection, the formulation may alternatively be stored in a cooler environment or include a viscosity modifier to maintain suspension of the radiopaque particles during storage.

In some embodiments, the pharmaceutical embolizing agent is freeze-dried (that is, lyophilized) for shelf-stability. In some embodiments, shelf-stability of the pharmaceutical embolizing agent is improved by the addition of antioxidants to protect lipids from oxidation.

In alternate embodiments, the pharmaceutical embolizing agent is packaged as a two-component system comprising: (a) a gel-forming polymer composition comprising a therapeutic agent (for example, in embodiments, an anti-VEGF compound or an analgesic compound) dispersed or dissolved therein, and (b) a separate vial containing a radiopaque powder, such as tantalum. The gel-forming polymer composition constitutes the medical gel matrix of the pharmaceutical embolizing agent. The radiopaque powder is combined with the gel-forming polymer composition comprising the therapeutic agent immediately prior to use, thereby ensuring substantially uniform suspension of the radiopaque material within the medical gel at the time of injection.

On the day of use, re-dispersion techniques may be employed if settling has occurred. In embodiments, re-dispersion methods include: (a) manual shaking, such as vigorous shaking or inversion of a vial or syringe for a specified duration (for example, approximately 1-2 minutes); (b) use of a mechanical mixer, such as a wrist-action shaker or laboratory rotator for several minutes; (c) syringe pull-push techniques, wherein the suspension is repeatedly drawn into a syringe and expelled back into the vial to disrupt sediment accumulation; and (d) use of an ultrasonic bath or ultrasonic cleaner to disperse powders. These techniques promote restoration of substantially uniform radiopacity prior to injection.

Thus, the pharmaceutical embolizing agent of the present specification is configured to demonstrate sufficient yield stress to prevent reflux, controlled setting kinetics following deployment, uniform radiopacity for real-time imaging, and compatibility with selected catheter dimensions and injection techniques. The rheology of the pharmaceutical embolizing agent, catheter selection, injection parameters, and procedural technique are balanced to achieve safe and effective embolization.

### Medical Gels for Use in the Pharmaceutical Embolizing Agent

In some embodiments, the injectable medical-grade gel constituting the pharmaceutical embolizing agent of the present specification comprises a biocompatible hydrogel, polymeric embolic, or hemostatic agent. The gel is configured to serve one or more therapeutic and procedural functions, including: (a) embolization, such as blocking blood flow to treat aneurysms, tumors, or vascular malformations; (b) drug delivery, including controlled release of therapeutics within blood vessels; (c) vessel sealing and hemostasis, such as stopping arterial bleeding after catheterization; and (d) aneurysm treatment, including use as an adjunct to endovascular coiling to enhance occlusion.

Hydrogels are highly biocompatible, water-swollen polymer networks that can be injected and solidify inside arteries. Some are bioresorbable, while others are permanent. Example hydrogels are: polyethylene glycol (PEG)-based hydrogels, polyvinyl alcohol (PVA)-based hydrogels, alginate-based hydrogels, chitosan-based hydrogels, hyaluronic acid (HA) hydrogels and fibrin-based hydrogels. It should be noted herein that while a list of hydrogels has been provided, this list is not exhaustive. Further, it should be noted that the hydrogels that are contemplated for use in the present specification should be non-permanent. In embodiments, a non-permanent hydrogel is used to support repeat treatment for the patient.

The pharmaceutical embolizing agent of the present specification is formulated to minimize induction of thrombotic or fibrotic vascular wall response, thereby preserving the recanalization potential of the target artery following gel resorption. This is achieved through several formulation characteristics: (a) hyaluronic acid is a native component of the endothelial glycocalyx with established anti-thrombogenic properties, minimizing platelet activation and intimal hyperplasia compared to foreign polymer embolic agents; (b) absence of dimethyl sulfoxide (DMSO), which is present in Onyx and Squid and causes endothelial toxicity and necrosis at the vessel wall, promoting fibrosis and permanent occlusion; and (c) non-adhesive gel-to-vessel-wall interaction, preventing mechanical injury to the endothelium during deployment. In hyaluronic acid-based embodiments, on-demand dissolution of the gel may be achievable through intra-arterial administration of exogenous hyaluronidase, which enzymatically degrades the hyaluronic acid backbone. This potential reversibility mechanism, while requiring further clinical validation, is not available with any existing permanent embolic agent and represents a theoretical safety advantage, particularly in the event of inadvertent non-target embolization or procedural complication. In non-HA-based embodiments, recanalization relies on passive hydrolytic or enzymatic degradation of the gel matrix, supported by the favorable vascular biology of the MMA described herein and by published clinical evidence of recanalization following temporary embolization of the MMA with absorbable agents such as gelatin sponge.

In some embodiments, the pharmaceutical embolizing agents of the present specification are formulated using hydrogels that remain flowable under high shear conditions, such as during catheter-based injection, but rapidly increase in viscosity or structurally recover once delivered into tissue or a target vessel, thereby helping to “lock” the incorporated therapeutic agents in place.

Polymeric embolic agents are injectable liquid embolization agents that polymerize upon injection, forming a solid plug inside the artery. Examples of polymeric embolic agents include ethylene-Vinyl Alcohol (EVOH) polymers, acrylic polymer gels and cyanoacrylate-based gels.

Hemostatic and vessel-sealing gels are used to control arterial bleeding or seal puncture sites after catheterization. Exemplary hemostatic and vessel-sealing gels may include fibrin glue, collagen-gelatin hemostatic agents, chitosan-based hemostatic agents, and polyethylene glycol (PEG)-based sealants.

Additional types of injectable arterial gels include self-healing hydrogels, bioresorbable hydrogels, gene therapy-loaded gels, and magnetically controlled gels.

In some embodiments, the medical gel comprises PVA-based microspheres mixed with a contrast media (for example, iodine-based) for radio visibility.

In some embodiments, the medical gel comprises PLGA (poly lactic-co-glycolic acid) microspheres or depots.

In some embodiments, the medical gel incorporates temperature-responsive or pH-responsive polymers to transition to a solid-like depot in the artery for controlled drug (bevacizumab or bupivacaine) release. Thermosensitive sol-gel transition polymers, including PEG-based copolymers, poloxamer systems, and chitosan/β-glycerophosphate hydrogels, remain liquid under cooled or low-shear conditions and transition to a gel state at physiological temperature (~37° C.). Such phase-transition systems enable injection followed by in situ depot formation.

In embodiments, the medical gel is configured to exhibit biocompatibility and favorable in vivo performance characteristics. The gel minimizes thrombogenicity, prevents unintended migration, and maintains arterial patency where clinically appropriate. Further, the gel is characterized by remaining substantially non-adhesive to delivery devices and vascular walls, thereby facilitating controlled deployment and potential catheter retrieval. The gel is also formulated to be non-settling, such that its constituents remain substantially uniformly distributed during storage and delivery.

In accordance with embodiments of the present specification, the gel is compatible with dimethyl sulfoxide (DMSO)-based delivery systems or, alternatively, configured for solvent-free designs, thereby enabling flexibility in formulation and clinical use.

In some embodiments, following biocompatible hydrogels can serve as bevacizumab and bupivacaine carriers as per Table 1 below.

TABLE 1

| Gel Type | Advantages |
|---|---|
| PEG-PLGA Based | Highly biocompatible, tunable degradation, is liquid at room |
| Thermoresponsive Hydrogels | temperature (~25° C.), and gels at body temperature (~37° C.). |
| PLGA micro-particles | Micro-particles or microspheres release bevacizumab or |
| | bupivacaine slowly over 2-4 weeks |
| Chitosan/β-glycerophosphate | Bioactive, supports wound healing, non-thrombogenic |
| Based Hydrogels | |
| HA (hyaluronic acid)-based gels | Enzymatically degradable via endogenous hyaluronidase; |
| | recanalization timeline tunable by crosslink density; |
| | inherently anti-thrombogenic (HA is native component of |
| | endothelial glycocalyx); chemically inert to both |
| | bevacizumab and bupivacaine; non-adhesive to vessel wall; |
| | on-demand dissolution with exogenous intra-arterial |
| | hyaluronidase provides unique reversibility not available with |
| | any existing embolic agent |
| HA (hyaluronic acid) | Improve tissue adherence and drug retention |
| poloxamer hybrid gels | |
| PEG-chitosan hybrid hydrogel | PEG ensures slow degradation, chitosan supports dura |
| | healing. Bevacizumab or bupivacaine is released over 10-20 |
| | days for sustained VEGF inhibition. |
| Fibrin-Based Hydrogels | Natural clotting ability, good tissue integration |
| Alginate Hydrogels | Biodegradable, tunable porosity |
| PLGA Microsphere Gels | Extended release (10-30 days), FDA-approved for drug delivery |
| (Poly(lactic-co-glycolic acid)) | |

The First Composition (A Sustained-Release Hydrogel Formulation) Used for the Treatment of cSDH

In some embodiments, the pharmaceutical embolizing agent has a first composition comprising a medical gel incorporating a sustained-release anti-VEGF therapeutic such as, for example, bevacizumab (Avastin®). In various embodiments, the anti-VEGF therapeutic is selected from a group consisting of bevacizumab, ranibizumab, aflibercept, and biosimilars or functional equivalents thereof, or any other agent that inhibits vascular endothelial growth factor signaling. Bevacizumab is a preferred anti-VEGF therapeutic due to its established clinical use, large molecular weight (approximately 149 kDa) which slows diffusion through the gel matrix and supports sustained local retention, and its well-characterized anti-neovascular activity relevant to the pathological neoangiogenesis of subdural neo-membranes in cSDH.

In embodiments, the first composition is administered to a patient for the treatment of cSDH, and in particular may be delivered via middle meningeal artery (MMA) injection. In embodiments, the medical gel is a biodegradable hydrogel encapsulating bevacizumab. In some embodiments, the medical gel is a biodegradable hydrogel with liposomal bevacizumab for an extended effect of double-sustained release since the liposomes release over a period of 72 hours and the hydrogel prolongs the release to 2 to 6 weeks. This composition is characterized by a synergy between the hydrogel matrix and the liposome bevacizumab encapsulation. In embodiments, the first composition exhibits tunable degradation kinetics optimized for subdural hematoma treatment, such that the formulated gel degrades over a period of 2 to 8 weeks and preferably, 2-6 weeks, consistent with the double-sustained-release mechanism of liposomal bevacizumab within the hydrogel matrix while maintaining sufficient structural integrity to achieve embolization. When delivered through MMA injection, the first composition reduces cSDH recurrence by stabilizing fragile neo-membrane vasculature associated with the hematoma. The medical gel is configured to provide mechanical embolization of targeted vascular branches while simultaneously serving as a local drug delivery matrix for controlled release of the anti-VEGF therapeutic. Accordingly, the first composition provides a dual-action therapy comprising (i) mechanical embolization to reduce pathological blood flow and (ii) localized VEGF inhibition to modulate angiogenesis and vascular permeability, thereby addressing both the structural and biological components of cSDH pathophysiology.

Bevacizumab is a monoclonal antibody that inhibits vascular endothelial growth factor (VEGF), a key protein involved in angiogenesis (the formation of new blood vessels). In the context of cSDH, the pathological overexpression of VEGF within the dura mater drives the formation of fragile, hyperpermeability neo-membranes supplying the hematoma. By blocking VEGF signaling, bevacizumab inhibits this pathological neoangiogenesis, reducing vascular permeability of the neo-membrane capillaries and thereby interrupting the cycle of recurrent bleeding and hematoma re-accumulation.

### Dosage and Local Concentration

The present specification aims at reducing the total overall dosage of bevacizumab by administering it in a high locally sustained concentration. In embodiments, the first composition comprises less than approximately 10% by weight of the bevacizumab, preferably less than approximately 5%, and more preferably less than approximately 2% by weight of the bevacizumab dissolved or dispersed therein, such that the presence of the therapeutic agent has minimal effect on injectability, flow behavior, and catheter-based delivery performance.

The local concentration of bevacizumab required for efficacy depends on its application and target tissue exposure. Below is a summary Table 2 providing estimated effective concentrations in certain applications.

TABLE 2

| | | Local Effective | | Minimum Effective | |
|---|---|---|---|---|---|
| Indication | Target Tissue | Concentration (μg/mL) | | Concentration (MEC) | |
| Cancer (intravenous) | Tumor Microenvironment | 1-10 | μg/mL | ~0.5-2 | μg/mL |
| Ophthalmology | Vitreous Humor | 25,000 μg/mL (peak), >1 | | ~0.1-0.5 | μg/mL |
| (Intravitreal) | | μg/mL (sustained) | | | |
| Systemic Plasma | Blood Circulation | 50-100 | μg/mL | ~5-10 | μg/mL |

(intravenous dosing)

Following is an estimated local drug retention and concentration based on infusion volume, flow dynamics, and tissue binding:

Dosing for MMA infusion:
Concentration: 1-10 μg/mL in dura over 1-2 weeks.
Injection Volume: 0.5-1.5 ml bevacizumab solution.
Infusion Rate: Slow infusion over ~10-15 min to allow tissue uptake.
Dose Range: 2.5 mg-5 mg bevacizumab per MMA injection.

Expected tissue concentration:
Peak local concentration (within dura): ~50-200 μg/mL immediately post-infusion.
Steady-state diffusion into hematoma capsule: 1-10 μg/mL over ~1-2 weeks.
Elimination from dura: via lymphatic drainage and venous outflow.
   The Second Composition (A Sustained-Release Hydrogel Formulation) for the Treatment of Headaches. Intractable Migraines and Post-Craniotomy Pain or Post-Operative Pain

In some embodiments, the pharmaceutical embolizing agent has a second composition comprising a medical gel mixed with a sustained-release analgesic therapeutic such as, for example, bupivacaine. The second composition is administered to a patient to treat headaches, intractable migraines and post-craniotomy pain or post-operative pain.

In various embodiments, the second composition is administered to a patient via MMA injection for the treatment of a plurality of conditions, including chronic migraine, cluster headache, and post-craniotomy or other post-operative pain. The second composition is configured to provide long-lasting, localized therapy targeted to cranial nerve-associated pathways supplied by branches of the MMA.

In current therapeutic approaches, lidocaine is commonly administered. However, its pharmacokinetic profile presents limitations distinct from those associated with liquid embolic agents of the present specification. Upon intra-arterial administration, a substantial portion of lidocaine may rapidly traverse the target vascular bed and pass into the venous circulation, thereby limiting localized retention and reducing therapeutic efficiency at the intended site of action. Moreover, systemic exposure to lidocaine at higher doses may produce dose-dependent adverse effects, including potential impacts on cardiac conduction and myocardial function. Accordingly, the transient intravascular residence time and risk of systemic cardiotoxicity associated with lidocaine highlight limitations in its use for sustained, localized vascular therapy.

In some embodiments, the second composition comprises a bupivacaine-loaded thermosensitive hydrogel formulated for sustained intravascular residence and controlled release. Upon injection into the patient's MMA, the thermosensitive hydrogel undergoes in situ structural transition and provides prolonged release of bupivacaine over a period ranging from approximately 2 to 6 weeks, thereby enabling extended analgesic effect while minimizing systemic exposure.

In embodiments, the analgesic mechanism of the second composition operates through two sequential and complementary phases. During an initial high-concentration phase (approximately days 1 through 14 post-injection), peak bupivacaine release from the liposomal carrier provides free drug concentrations sufficient for sodium-channel-mediated conduction block of perivascular trigeminal afferents supplying branches of the MMA. This acute phase provides direct analgesic benefit and may induce post-block analgesia, a well-documented phenomenon in which temporary nerve conduction block produces pain relief substantially outlasting the pharmacokinetic duration of the drug, likely through resetting of central sensitization of the trigeminovascular system. During a subsequent sustained low-concentration phase (approximately weeks 2 through 6), sub-block concentrations of bupivacaine exert anti-neuroinflammatory effects independent of sodium channel blockade, including inhibition of substance P and calcitonin gene-related peptide (CGRP) release from perivascular nociceptors, and suppression of trigeminovascular activation. This dual-phase mechanism distinguishes the second composition from simple analgesic depots that rely solely on conduction block, and supports the claimed extended therapeutic window of 2 to 6 weeks.

In embodiments, the medical gel is a biodegradable hydrogel, encapsulating bupivacaine, to avoid permanent embolization.

In some embodiments, the second composition comprises a combination of a hydrogel with liposomal bupivacaine for an extended effect of double-sustained release since the liposomes release over 72 hours and the hydrogel prolongs the release to 2 to 6 weeks. This composition is characterized by a synergy between the hydrogel matrix and the liposome bupivacaine encapsulation.

### Dosage and Local Concentration

In some embodiments, a dose of 30 to 60 milligram (mg) bupivacaine in liposomal form is used with a medical gel of a matching embolic volume of approximately 0.5 to 2 mL. The concentration may be increased to 30-50 mg/mL for a localized and a controlled delivery. The liposomal formulation is stable during use, is pH-neutral, and is available, in some embodiments, in size of approximately 100 nm.

Approaches to formulate high-concentration bupivacaine in small volumes require deliberate and careful considerations to stabilize the drug. In one technique, bupivacaine is used in a free base. The free base is much less water-soluble than the HCl salt, which helps prevent it from dissolving immediately upon injection (thus avoiding a large bolus effect).

In embodiments, the second composition comprises less than approximately 10% by weight of the bupivacaine, preferably less than approximately 5%, and more preferably less than approximately 2% by weight of the bupivacaine dissolved or dispersed therein, such that the presence of the therapeutic agent has minimal effect on injectability, flow behavior, and catheter-based delivery performance.

The pharmaceutical embolizing agent of the present specification releases bupivacaine at a controlled rate such that systemic plasma concentrations at any given time remain substantially below the toxic threshold of approximately 2.5 mg/kg associated with cardiovascular and central nervous system toxicity. The total encapsulated dose of bupivacaine (30-60 mg) is within the range approved for regional anesthesia applications, and is consistent with precedent established by liposomal bupivacaine formulations approved for clinical use, which permit total doses substantially higher than equivalent free drug formulations. The controlled-release matrix ensures that peak systemic exposure is substantially lower than would result from equivalent bolus administration. Furthermore, the MMA is a small-caliber end artery with limited direct systemic venous communication, reducing the risk of rapid systemic absorption. In further embodiments, other long-acting local anesthetic agents, including ropivacaine, levobupivacaine, or other amide-class local anesthetic agents, may be employed as the analgesic agent in place of bupivacaine. Such agents share the property of prolonged sodium channel blockade at perivascular nerve terminals but may offer distinct safety or pharmacokinetic profiles suited to particular patient populations or clinical contexts.

### A System for Administering the Pharmaceutical Embolizing Agent

In embodiments, the pharmaceutical embolizing agent, formulated as either the first composition or the second composition, is administered by injecting the agent for controlled distal penetration within a target vascular territory, such as the middle meningeal artery (MMA). In some embodiments, the system for administering the pharmaceutical embolizing agent comprises a micro-catheter having an inner lumen.

In embodiments, the pharmaceutical embolizing agent is configured for injection through microcatheters having inner diameters ranging from approximately 0.0135 to 0.0314 inches (equivalent to approximately 0.3 mm to 0.8 mm) and lengths ranging from approximately 140 to 167 cm (and, in one preferred embodiment, a length of 150 cm and, in another preferred embodiment, a length of 160 cm). In one embodiment, the pharmaceutical embolizing agent is injectable through a microcatheter having an inner diameter of 0.017 inches (corresponding to 0.4318 mm, and radius of 0.2159 mm).

In embodiments, upon delivery, the pharmaceutical embolizing agent is capable of travelling into MMA branches that are defined by a region that has a size that can extend 0.1 cm to 25 cm, and every numerical increment therein, from the injection site within the target artery. In some other embodiments, upon delivery, the pharmaceutical embolizing agent is capable of traveling approximately 5 to 25 centimeters into distal MMA branches from a site of injection while avoiding capillary transit into the venous circulation. The injection volume may range from approximately 0.5 to 2.0 mL. In embodiments, the pharmaceutical embolizing agent is injected at a rate ranging from approximately 0.01 to 0.05 mL/s using manual injection under injection pressures of less than or equal to 300 mmHg (approximately 4 psi) and not exceeding the mean arterial perfusion pressure of the target artery. These pressure and flow parameters are selected to achieve the targeted penetration distance of at least 5 to 25 cm within MMA branches while permitting controlled delivery without operator fatigue. The rheological profile of the pharmaceutical embolizing agent ensures injectability, facilitates distal navigation, and promotes plug formation under low-flow conditions.

The distal penetration depth of the pharmaceutical embolizing agent is of particular importance for the headache and intractable migraine indications. The trigeminal perivascular innervation relevant to migraine pathophysiology, including the trigeminovascular afferents implicated in cortical spreading depression and meningeal neurogenic inflammation, is densest in the distal meningeal branches and arterioles of the MMA, beyond the proximal trunk. Proximal MMA embolization, as employed for cSDH hemostasis, may be insufficient to achieve perivascular contact between the bupivacaine-containing second composition and the relevant trigeminal nerve terminals. Accordingly, for the headache indication, the rheological profile of the pharmaceutical embolizing agent—specifically the low yield stress (5 to 20 Pa) and strong shear-thinning behavior—is specifically designed to enable distal penetration into second- and third-order meningeal branches under modest injection pressure, achieving perivascular drug deposition in the trigeminal-innervated distal meningeal territory that is the primary therapeutic target for migraine treatment. In some embodiments, for the headache indication, the pharmaceutical embolizing agent is injected to achieve penetration of at least 5 cm, and preferably 15 to 25 cm, beyond the main MMA trunk into distal meningeal branches, as confirmed by fluoroscopic visualization of the radiopaque gel. It should be noted that in embodiments, the penetration area is defined by a region that has a size that can extend 0.1 cm to 25 cm, and every numerical increment therein, from the injection site within the target artery.

Following injection, the pharmaceutical embolizing agent structurally stabilizes within approximately 30 seconds to 5 minutes, thereby promoting localized retention within distal vascular branches. Preferably, the pharmaceutical embolizing agent does not set during transit for an appreciable amount of time, but instead undergoes structural recovery after deposition to form a stable embolic plug.

In embodiments, reflux, defined as a condition in which the pharmaceutical embolizing agent exiting a distal end of a catheter travels backward over the distal end of the catheter rather than forward into distal circulation, may be mitigated through one or more reflux prevention techniques. In embodiments, reflux prevention techniques include: (a) use of a balloon-tipped catheter, wherein an inflatable balloon is expanded to prevent proximal backflow; (b) use of a catheter having a bulbous tip geometry; or (c) staged plug-formation techniques in which a small initial volume of the pharmaceutical embolizing agent is injected and permitted to solidify prior to full infusion, thereby forming a proximal barrier to reflux.

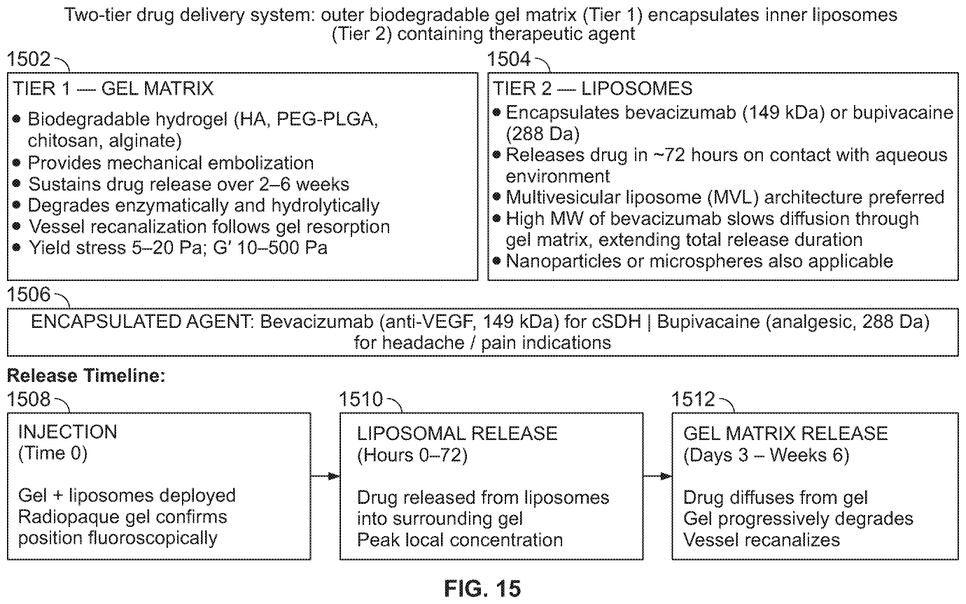

FIG. 15 illustrates a double sustained-release architecture for delivering a therapeutic agent using a two-tier drug delivery system, in accordance with some embodiments of the present specification. The system comprises an outer biodegradable gel matrix and an inner encapsulated carrier system, together enabling controlled release of a therapeutic compound over multiple time scales. The diagram shows the structural hierarchy and release sequences for the different functional layers and stages of drug delivery.

Referring to box 1502, the outer delivery or first tier component comprises a biodegradable hydrogel matrix that includes materials such as hyaluronic acid (HA), PEG-PLGA, chitosan, or alginate. This gel matrix provides mechanical embolization and serves as the primary structural scaffold for the delivery system. The gel matrix is configured to sustain drug release for approximately 2-6 weeks and undergoes degradation through enzymatic and hydrolytic processes. As the gel gradually resorbs, the previously embolized vessel may recanalize. The gel matrix may exhibit rheological properties including a yield stress of approximately 5 Pa to 20 Pa and a storage modulus (G′) in the range of approximately 100 Pa to 500 Pa, enabling it to maintain structural stability while allowing catheter-based injection.

Referring to box 1504, the inner delivery or second tier component comprises liposomal carriers encapsulated within the outer gel matrix. These liposomes contain the therapeutic agent and provide an additional controlled-release layer. The liposomal system is used, in embodiments, to encapsulate agents such as bevacizumab (approximately 149 kDa) or bupivacaine (approximately 288 Da). Upon exposure to an aqueous physiological environment, the liposomes may release the drug over an initial period of approximately 72 hours. In some embodiments, a multivesicular liposome (MVL) architecture is employed to further regulate release kinetics. Because bevacizumab has a relatively high molecular weight, its diffusion through the surrounding gel matrix may be slowed, thereby extending the overall release duration. Alternative encapsulation formats such as nanoparticles or microspheres can also be employed within the gel matrix.

Box 1506 indicates the therapeutic payload contained within the liposomal carriers. In some embodiments, the payload comprises bevacizumab, an anti-VEGF agent used for chronic subdural hematoma (cSDH) indications. In other embodiments, the payload may comprise bupivacaine, an analgesic agent used for headache or pain-related indications. The architecture therefore supports different therapeutic agents depending on the intended clinical application.

The lower portion of FIG. 15 illustrates a release timeline through three sequential boxes that represent the stages of drug delivery following administration. Referring to the box 1508 (at time 0), the gel matrix containing the liposomes is delivered to the target location. At this stage, the radiopaque gel allows fluoroscopic confirmation of accurate placement within the vasculature.

Referring to the box 1510 (at time=0 hours to 72 hours), the therapeutic agent is initially released from the liposomes into the surrounding gel matrix. During this phase, the drug diffuses into the adjacent tissue environment, producing a peak local concentration shortly after administration.

Finally, referring to the box 1512 (at time=3 days to 6 weeks), the gel matrix gradually releases the remaining drug as it undergoes progressive degradation. During this extended phase, the therapeutic agent continues to diffuse from the gel into surrounding tissues, maintaining sustained local exposure. As the biodegradable gel matrix is resorbed, the previously embolized vessel may recanalize, thereby completing the delivery cycle.

### Methods of Preparing and Administering the Pharmaceutical Embolizing Agent (First Composition or Second Composition)

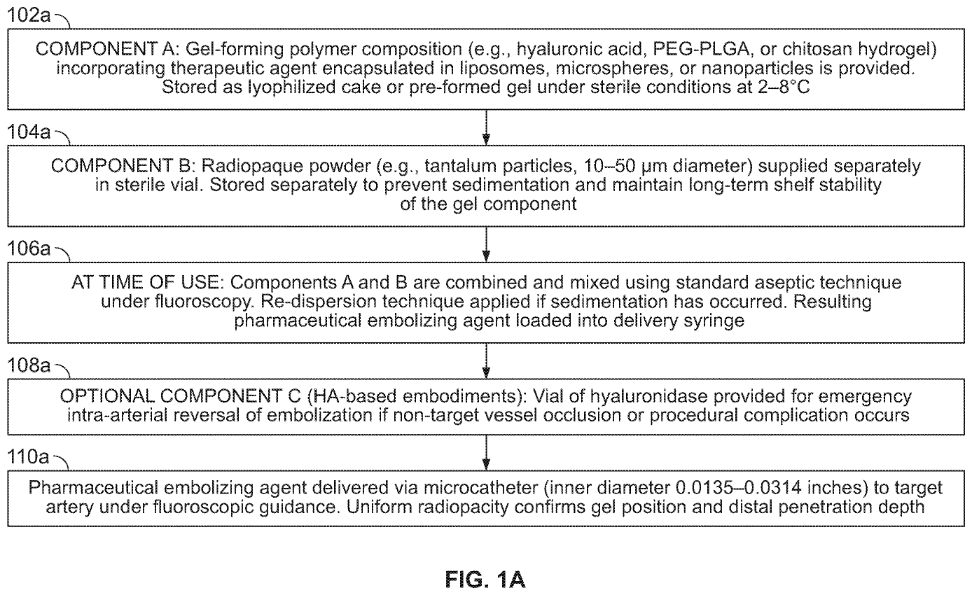

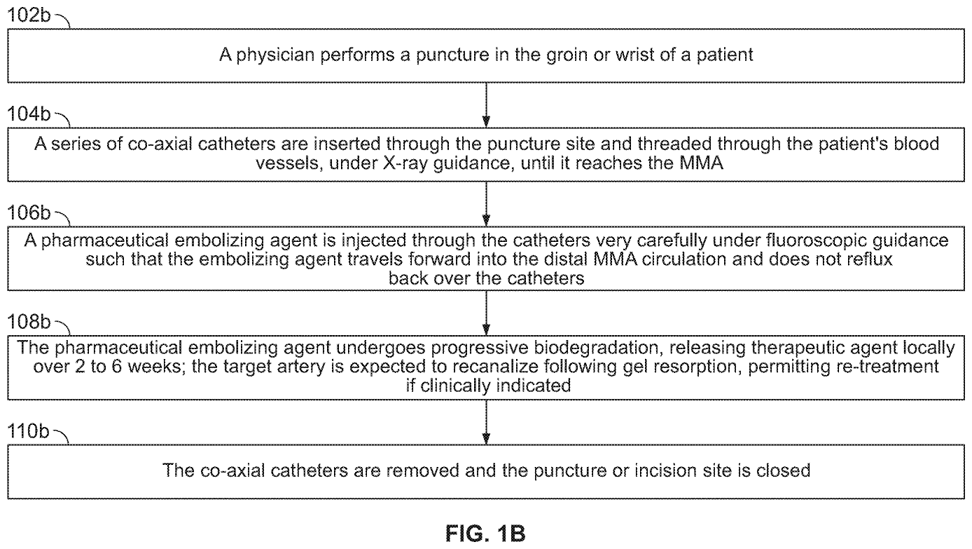

FIG. 1A is a flowchart describing exemplary steps for preparation and administration of a pharmaceutical embolizing agent to treat a condition, in accordance with some embodiments of the present specification. At step 102a, a gel-forming polymer composition (Component A) is provided. In some embodiments, the gel-forming polymer comprises materials such as hyaluronic acid, PEG-PLGA, or chitosan hydrogel. The composition incorporates a therapeutic agent (such as, for example, bevacizumab or bupivacaine) encapsulated within carriers such as liposomes, microspheres, or nanoparticles. Component A may be stored as a lyophilized cake or as a pre-formed gel under sterile conditions, for example at temperatures between approximately 2-8° C., to preserve stability prior to use.

At step 104a, a radiopaque component (Component B) is provided separately. In some embodiments, the radiopaque component comprises a radiopaque powder such as tantalum particles having a diameter of approximately 10-50 μm. Component B is supplied in a separate sterile vial and stored independently from the gel component to prevent sedimentation and to maintain long-term stability of the polymer composition.

At step 106a, at the time of use, Components A and B are combined and mixed using standard aseptic techniques, typically under fluoroscopic guidance. If sedimentation of the radiopaque particles has occurred during storage, a re-dispersion technique may be applied to restore uniform distribution of the radiopaque material within the gel matrix. The resulting mixture forms the pharmaceutical embolizing agent, which is then loaded into a delivery syringe or similar administration device.

At step 108a, in certain embodiments, an optional Component C is provided. Component C may comprise a vial containing hyaluronidase or another enzymatic agent capable of degrading the gel matrix. In embodiments where the gel matrix is hyaluronic acid-based, the hyaluronidase may be used for emergency intra-arterial reversal of embolization in the event of non-target vessel occlusion or other procedural complications. Hyaluronidase is administered intra-arterially to the embolized vessel at a dose sufficient to enzymatically degrade the hyaluronic acid gel matrix, thereby restoring blood flow through the target artery.

At step 110a, the prepared pharmaceutical embolizing agent is delivered to a target artery (for example, MMA) using a microcatheter. In some embodiments, the microcatheter has an inner diameter of approximately 0.0135-0.0314 inches. Delivery is performed under fluoroscopic guidance, and the radiopaque component enables visualization of the embolizing gel during injection. Uniform radiopacity allows the operator to confirm correct positioning of the gel and to monitor distal penetration depth within the target vasculature.

In some embodiments, repeat treatment of the condition may be performed in a patient who has previously undergone a target artery embolization with a first pharmaceutical embolizing agent. The repeat treatment comprises confirming recanalization of the previously embolized target artery using an imaging modality and administering a second pharmaceutical embolizing agent through a microcatheter to the recanalized artery. In some embodiments, the interval between administration of the first pharmaceutical embolizing agent and the second pharmaceutical embolizing agent is at least about 4 weeks. In other embodiments, the interval between administration of the first pharmaceutical embolizing agent and the second pharmaceutical embolizing agent ranges from about 4 weeks to about 24 weeks.

In some embodiments, a kit for preparing a pharmaceutical embolizing agent is provided. The kit comprises a first component including a gel-forming polymer composition incorporating a sustained-release therapeutic agent encapsulated in liposomes, microspheres, or nanoparticles, and a second component including a radiopaque powder, wherein the first component and the second component are stored separately and are combined immediately prior to intra-arterial administration. In some embodiments, the radiopaque powder comprises tantalum particles. In certain embodiments, the kit further comprises a microcatheter sized for intra-arterial delivery. In other embodiments, the kit further comprises a vial of hyaluronidase configured for emergency reversal of embolization.

FIG. 1B is a flowchart describing exemplary steps of a method of administering the pharmaceutical embolizing agent to treat a patient's condition, in accordance with some embodiments of the present specification. In embodiments, the pharmaceutical embolizing agent is formulated as either the first composition or the second composition. In embodiments, the patient's condition may include cSDH, headaches, intractable migraines, post-craniotomy pain or post-op pain such as post-op spine and knee pain, and low back pain.

At step 102, a physician performs a puncture in the groin or wrist of the patient.

At step 104, a series of co-axial catheters are inserted through the puncture site and threaded through the patient's blood vessels, under X-ray guidance, until it reaches the MMA—as is standard practice and well known to persons of ordinary skill in the art.

At step 106, the pharmaceutical embolizing agent is injected through the catheter(s) very carefully under fluoroscopic guidance such that the embolizing agent travels forward into the distal MMA circulation and does not reflux back over the catheter(s). Additional technologies to prevent reflux—such as, for example, a balloon tipped catheter, may be used.

It should be appreciated that a purpose of the injection is to allow for (slow release) delivery of the pharmaceutical embolizing agent distally into the MMA bed while reducing the likelihood of drug toxicity and/or adverse effects from the drug going in a wrong artery. Depending on the patient's underlying condition, different compositions of the pharmaceutical embolizing agent may be used.

At step 108, the pharmaceutical embolizing agent undergoes progressive biodegradation, releasing the therapeutic agent locally over 2 to 6 weeks. In embodiments, the target artery is expected to recanalize following gel resorption, permitting re-treatment if clinically indicated.

Finally, at step 110, the co-axial catheters are removed and the puncture or incision site is closed.

### Exemplary Use Case Scenarios

A Use Case for Treating cSDH (chronic Subdural Hematoma)

Chronic subdural hematoma (cSDH) is characterized by the accumulation of blood and associated inflammatory products within the subdural space. cSDH is frequently associated with neurological deficits, cognitive impairment, persistent headaches, intractable migraines, and prolonged post-craniotomy or post-operative pain.

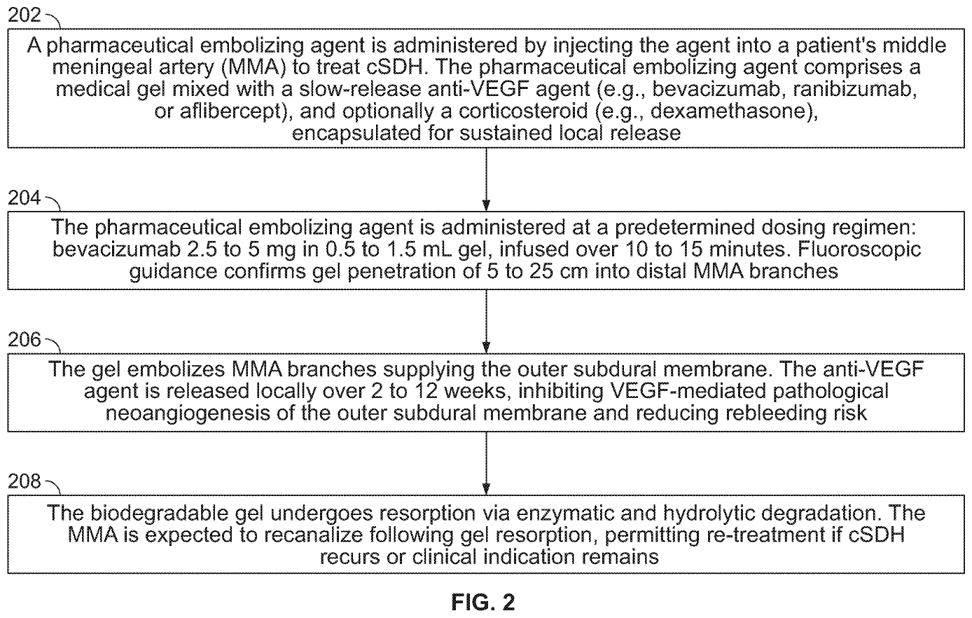

FIG. 2 is a flowchart describing exemplary steps of a method of treating cSDH, in accordance with some embodiments of the present specification. At step 202, a pharmaceutical embolizing agent is administered by injecting the agent into a patient's middle meningeal artery (MMA) in order to treat cSDH.

In some embodiments, the pharmaceutical embolizing agent comprises a medical gel mixed with a slow-release anti-VEGF agent (e.g., bevacizumab, ranibizumab, or aflibercept), and optionally a corticosteroid (e.g., dexamethasone), encapsulated for sustained local release.

At step 204, the pharmaceutical embolizing agent is administered at a predetermined dosing regimen configured for an expected local tissue concentration. Fluoroscopic guidance confirms gel penetration of 5 to 25 cm into distal MMA branches.

In some embodiments, the dosing regimen and expected local concentration is as follows:

Dosing for MMA infusion:
Concentration: 1-10 μg/mL in dura over 1-2 weeks.
Injection Volume: 0.5-1.5 ml bevacizumab solution.
Infusion Rate: Slow infusion over ~10-15 min to allow tissue uptake.
Dose Range: 2.5 mg-5 mg Bevacizumab per MMA injection.

Expected tissue concentration:
Peak local concentration (within dura): ~50-200 μg/mL immediately post-infusion.
Steady-state diffusion into hematoma capsule: 1-10 μg/mL over ~1-2 weeks.
Elimination from dura: via lymphatic drainage and venous outflow.

At step 206, the medical gel embolizes MMA branches supplying the outer subdural membrane. The anti-VEGF agent is released locally over 2 to 12 weeks, and in some embodiments 2 to 6 weeks, inhibiting VEGF-mediated pathological neoangiogenesis of the outer subdural membrane and reducing rebleeding risk.

At step 208, the medical gel, being biodegradable, undergoes resorption via enzymatic and hydrolytic degradation. The MMA is expected to recanalize following gel resorption, permitting re-treatment if cSDH recurs or clinical indication remains.

### Therapeutic Endpoints

In one embodiment, an average decrease in self-reported patient pain scores by at least 30% is accompanied by an occurrence of pain associated with cSDH (such as, for example, headache, migraine-like pain, post-craniotomy pain, post-operative pain, neuropathic pain), of less than 5%, preferably less than 3%, more preferably less than 2% and most preferably less than 1%.

As described earlier, patient pain may be assessed using standardized pain assessment tools such as, for example, Visual Analog Scale (VAS), Numeric Rating Scale (NRS), Verbal Rating Scale (VRS), McGill Pain Questionnaire (MPQ), Oswestry Disability Index (ODI), Knee Injury and Osteoarthritis Outcome Score (KOOS), Brief Pain Inventory (BPI), Headache Impact Test (HIT-6) and Migraine Disability Assessment (MIDAS).

Using the embodiments of the present specification, the average decrease in pain scores (generated using one or more of the standardized pain assessment tools) in patients diagnosed with cSDH is at least 30% of the pre-treatment pain score. In some cases the average decrease in pain scores is at least 15%, and in yet some other cases, the average decrease in pain scores is at least 20%.

### A Use Case Scenario for Treating Headaches, Intractable Migraine Conditions and Post-Craniotomy Pain

Headaches, intractable migraine and post-craniotomy pain or post-op pain conditions represent prevalent and often debilitating neurological disorders characterized by recurrent or persistent craniofacial pain that significantly impairs quality of life. While headaches may be vascular in origin, intractable migraine refers to a severe, treatment-resistant subtype marked by intense, throbbing pain often accompanied by nausea, and abnormal sensitivities to light and sound. These conditions are frequently chronic, inadequately managed by conventional pharmacological or interventional therapies, and impose substantial functional, psychological, and socioeconomic burdens on affected patients.

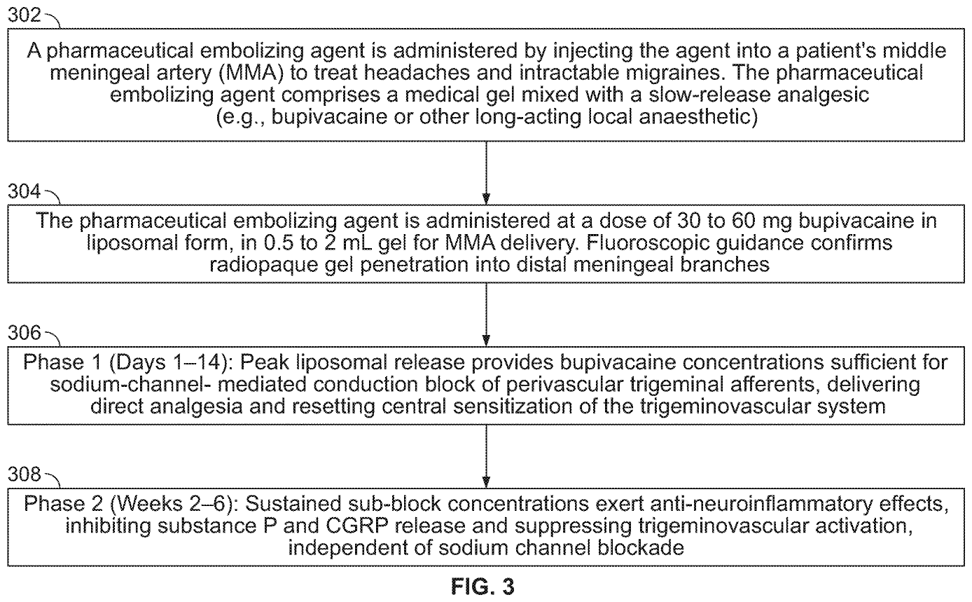

FIG. 3 is a flowchart describing exemplary steps of a method of treating headaches, intractable migraine and post-craniotomy pain, in accordance with some embodiments of the present specification. At step 302, a pharmaceutical embolizing agent is administered by injecting the agent into a patient's middle meningeal artery (MMA) in order to treat headaches and intractable migraines.

In some embodiments, the pharmaceutical embolizing agent comprises a medical gel mixed with a slow-release analgesic—such as, for example, bupivacaine or other long-acting local anaesthetic.

At step 304, the pharmaceutical embolizing agent is administered at a predetermined dosing regimen configured for an expected local tissue concentration. Fluoroscopic guidance confirms radiopaque gel penetration into distal meningeal branches.

In some embodiments, a dose of 30 to 60 milligram (mg) bupivacaine in liposomal form is used with the medical gel of a matching embolic volume of approximately 0.5 to 2 mL. The concentration may be increased to 30-50 mg/mL for a localized and a controlled delivery.

The low yield stress (5 Pa to 20 Pa) and shear-thinning profile of the agent enables distal penetration of at least 5 cm, and preferably 15 cm to 25 cm, beyond the main MMA trunk into second- and third-order meningeal branches, achieving perivascular contact with trigeminal afferents innervating the distal meningeal territory.

At step 306, in phase 1 (days 1-14), peak liposomal release provides bupivacaine concentrations sufficient for sodium-channel-mediated conduction block of perivascular trigeminal afferents, delivering direct analgesia and resetting central sensitization of the trigeminovascular system.

At step 308, in phase 2 (weeks 2-6), sustained sub-block concentrations exert anti-neuroinflammatory effects, inhibiting substance P and CGRP release and suppressing trigeminovascular activation, independent of sodium channel blockade.

### Therapeutic Endpoints

In one embodiment, an average decrease in self-reported patient pain scores by at least 30% is accompanied by an occurrence of pain associated with headaches, intractable migraines and post-craniotomy pain or post-op pain, of less than 5%, preferably less than 3%, more preferably less than 2% and most preferably less than 1%.

As described earlier, patient pain may be assessed using standardized pain assessment tools such as, for example, Visual Analog Scale (VAS), Numeric Rating Scale (NRS), Verbal Rating Scale (VRS), McGill Pain Questionnaire (MPQ), Oswestry Disability Index (ODI), Knee Injury and Osteoarthritis Outcome Score (KOOS), Brief Pain Inventory (BPI), Headache Impact Test (HIT-6) and Migraine Disability Assessment (MIDAS).

Using the embodiments of the present specification, the average decrease in pain scores (generated using one or more of the standardized pain assessment tools) in patients suffering from the conditions of headaches, intractable migraines and post-craniotomy pain is at least 30% of the pre-treatment pain score. In some cases the average decrease in pain scores is at least 15%, and in yet some other cases, the average decrease in pain scores is at least 20%.

A Use Case Scenario for Treating Pain Associated with Post-Operative Spine Pain and Knee Pain

Post-operative spine and knee pain is a multifactorial condition that may arise following surgical interventions such as laminectomy, discectomy, spinal fusion, arthroplasty, ligament reconstruction, or other reconstructive or decompressive procedures. The pain may result from a combination of tissue trauma, local inflammation, neural irritation, periosteal and capsular disruption, and postoperative scar formation. In the spine, post-operative pain may involve facet joints, paraspinal musculature, ligamentous structures, epidural tissues, or adjacent nerve roots, and may be associated with persistent inflammation, fibrosis, or altered segmental biomechanics. In the knee, post-surgical pain may involve synovial tissues, periarticular soft tissues, residual ligamentous strain, capsular tightness, or altered joint loading patterns following repair or replacement procedures. Patients commonly experience activity-related pain, stiffness, reduced range of motion, localized tenderness, and impaired functional capacity, which may interfere with rehabilitation and recovery.

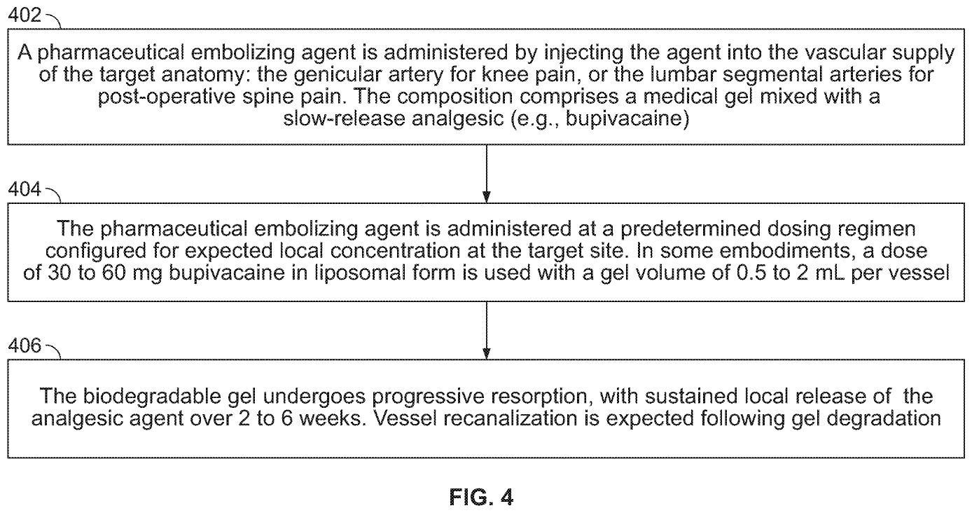

FIG. 4 is a flowchart describing exemplary steps of a method of treating post-operative spine and knee pain, in accordance with some embodiments of the present specification. At step 402, a pharmaceutical embolizing agent is administered by injecting the agent into a patient's vascular supply network to a target region/anatomy (that is, the genicular artery for knee pain, or the lumbar segmental arteries for post-operative spine pain) in order to treat post-operative knee and spine pain.

In some embodiments, the pharmaceutical embolizing agent comprises a medical gel mixed with a slow-release analgesic—such as, for example, bupivacaine.

In some embodiments, sites for injecting the pharmaceutical embolizing agent for treating post-operative spine pain are guided by the segmental arterial anatomy supplying the operated spinal level, including branches of the lumbar arteries arising from the abdominal aorta, the iliolumbar artery arising from the internal iliac artery, and, where applicable, lateral sacral branches supplying the lumbosacral region. Key vascular targets may include dorsal branches of the lumbar segmental arteries that supply the facet joints, paraspinal musculature, periosteum, and posterior ligamentous complex; radicular branches that accompany spinal nerve roots; and periarticular vascular networks supplying the zygapophyseal (facet) joints and adjacent soft tissues. In post-laminectomy or post-fusion settings, neovascularized scar tissue and inflamed peri-incisional soft tissues may also receive perfusion from these segmental and dorsal branches, rendering them clinically relevant targets for selective intra-arterial or perivascular delivery of the pharmaceutical embolizing agent to achieve localized vascular modulation and sustained analgesic release while minimizing non-target embolization of critical spinal cord or radiculomedullary arterial supply.

In some embodiments, sites for injecting the pharmaceutical embolizing agent for treating post-operative knee joint pain are guided by the genicular anastomosis, a vascular network primarily fed by branches of the popliteal artery with contributions from the femoral and anterior tibial arteries. Key targets include the superior (medial and lateral) genicular arteries supplying the upper capsule, the inferior (medial and lateral) genicular arteries supplying the lower capsule and patellar ligaments, and the middle genicular artery, which directly penetrates the joint capsule to supply the cruciate ligaments and synovium. These vessels also support the collateral ligaments, menisci (outer vascular zone), bones, and periarticular muscles, making them clinically relevant for targeted intra-arterial or perivascular pain modulation in knee joint disorders.

At step 404, the pharmaceutical embolizing agent is administered at a predetermined dosing regimen configured for an expected local tissue concentration.

In some embodiments, a dose of 30 to 60 milligram (mg) bupivacaine in liposomal form is used with the medical gel of a matching embolic volume of approximately 0.5 to 2 mL. The concentration may be increased to 30-50 mg/mL for a localized and a controlled delivery.

At step 406, the gel, being biodegradable, undergoes progressive resorption, with sustained local release of the analgesic agent over 2 to 6 weeks. Vessel recanalization is expected following gel degradation.

### Therapeutic Endpoints

In one embodiment, an average decrease in self-reported patient pain scores by at least 30% is accompanied by an occurrence of post-operative spine or knee pain, of less than 5%, preferably less than 3%, more preferably less than 2% and most preferably less than 1%.

As described earlier, patient pain may be assessed using standardized pain assessment tools such as, for example, Visual Analog Scale (VAS), Numeric Rating Scale (NRS), Verbal Rating Scale (VRS), McGill Pain Questionnaire (MPQ), Oswestry Disability Index (ODI), Knee Injury and Osteoarthritis Outcome Score (KOOS), Brief Pain Inventory (BPI), Headache Impact Test (HIT-6) and Migraine Disability Assessment (MIDAS).

Using the embodiments of the present specification, the average decrease in pain scores (generated using one or more of the standardized pain assessment tools) in patients suffering from post-operative spine or knee pain is at least 30% of the pre-treatment pain score. In some cases the average decrease in pain scores is at least 15%, and in yet some other cases, the average decrease in pain scores is at least 20%.

### A Use Case Scenario for Treating Low Back Pain

Low back pain is a highly prevalent musculoskeletal condition characterized by pain, stiffness, or discomfort localized to the lumbar region, often with variable radiation to the buttocks or lower extremities. It may arise from degenerative disc disease, facet joint pathology, muscular strain, ligamentous injury, or nerve root compression, and can present as acute, subacute, or chronic in nature. Associated symptoms may include restricted mobility, postural imbalance, and functional limitations that interfere with daily activities and quality of life.

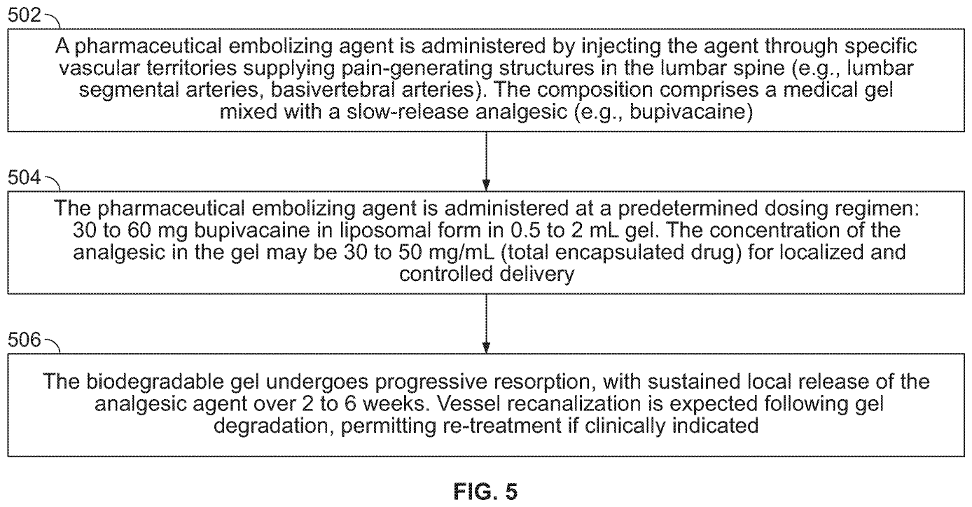

FIG. 5 is a flowchart describing exemplary steps of a method of treating low back pain, in accordance with some embodiments of the present specification. At step 502, a pharmaceutical embolizing agent is administered by injecting the agent through specific vascular territories supplying pain-generating structures in the lumbar spine (e.g., lumbar segmental arteries, basivertebral arteries).

In various embodiments, vascular territories supplying pain-generating structures comprise, for example, the lumbar segmental arteries (L2-L5) that provide dorsal branches to the facet joints, paraspinal muscles, and ligaments, as well as spinal branches to vertebral bodies, discs, dura, and nerve roots, making them direct routes to key pain sources. The iliolumbar artery supplies the L5-S1 facet and sacroiliac region, while the lateral sacral arteries serve the sacral facets, SI joint, and S1 root. Target zones for intra-arterial pain modulation include dorsal branches for facet and annulus-related pain, nutrient branches to discs and endplates, and sacroiliac territories, though care must be taken to avoid compromising spinal cord blood supply.

In some embodiments, the pharmaceutical embolizing agent comprises a medical gel mixed with a slow-release analgesic-such as, for example, bupivacaine.

At step 504, the pharmaceutical embolizing agent is administered at a predetermined dosing regimen configured for an expected local tissue concentration.

In some embodiments, a dose of 30 to 60 milligram (mg) bupivacaine in liposomal form is used with the medical gel of a matching embolic volume of approximately 0.5 to 2 mL. The concentration may be increased to 30-50 mg/mL (total encapsulated drug) for a localized and a controlled delivery.

At step 506, the medical gel, being biodegradable, undergoes progressive resorption, with sustained local release of the analgesic agent over 2 to 6 weeks. Vessel recanalization is expected following gel degradation, permitting re-treatment if clinically indicated.

### Therapeutic Endpoints

In one embodiment, an average decrease in self-reported patient pain scores by at least 30% is accompanied by an occurrence of low back pain, of less than 5%, preferably less than 3%, more preferably less than 2% and most preferably less than 1%.

As described earlier, patient pain may be assessed using standardized pain assessment tools such as, for example, Visual Analog Scale (VAS), Numeric Rating Scale (NRS), Verbal Rating Scale (VRS), McGill Pain Questionnaire (MPQ), Oswestry Disability Index (ODI), Knee Injury and Osteoarthritis Outcome Score (KOOS), Brief Pain Inventory (BPI), Headache Impact Test (HIT-6) and Migraine Disability Assessment (MIDAS).

Using the embodiments of the present specification, the average decrease in pain scores (generated using one or more of the standardized pain assessment tools) in patients suffering from low back pain is at least 30% of the pre-treatment pain score. In some cases the average decrease in pain scores is at least 15%, and in yet some other cases, the average decrease in pain scores is at least 20%.

### A Use Case Scenario for Treating Shoulder Pain and Rotator Cuff Tendinopathy

Chronic shoulder pain arising from rotator cuff tendinopathy, subacromial impingement syndrome, and calcific tendinitis is a highly prevalent musculoskeletal condition. A key pathological feature is the formation of abnormal neovasculature-accompanied by sensory nerve ingrowth-within normally avascular tendon tissue. These pathological neovessels express substance P and CGRP and represent a primary source of chronic pain in tendinopathic tissue. Transcatheter arterial embolization (TAE) targeting the abnormal neovascularization supplying the rotator cuff and subacromial bursa can achieve clinically meaningful pain reduction in patients with refractory subacromial impingement syndrome and calcific tendinitis.

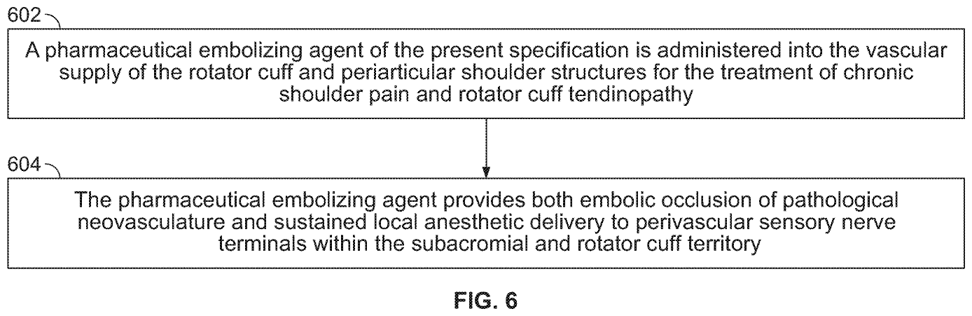

FIG. 6 is a flowchart describing exemplary steps of a method of treating shoulder pain and rotator cuff tendinopathy, in accordance with some embodiments of the present specification. At step 602, a pharmaceutical embolizing agent of the present specification is administered into the vascular supply of the rotator cuff and periarticular shoulder structures for the treatment of chronic shoulder pain and rotator cuff tendinopathy.

The primary arterial targets include branches of the anterior circumflex humeral artery supplying the supraspinatus and infraspinatus tendons, branches of the thoracoacromial artery (specifically the acromial and deltoid branches) supplying the subacromial bursa and superior rotator cuff, and the posterior circumflex humeral artery supplying the posterior cuff and teres minor. In calcific tendinitis, embolization is directed specifically at the neovasculature supplying the calcium deposit and adjacent reactive tissue.

In some embodiments, the pharmaceutical embolizing agent comprising the second composition (medical gel with sustained-release bupivacaine or other long-acting local anesthetic) is used in this setting.

At step 604, the pharmaceutical embolizing agent provides both embolic occlusion of pathological neovasculature and sustained local anesthetic delivery to perivascular sensory nerve terminals within the subacromial and rotator cuff territory. The dual mechanism of ischemic devascularization of pathological neovessels and sustained neuroinflammatory modulation at perivascular terminals is expected to produce durable pain relief substantially exceeding that achievable by neovascular embolization alone.

Therapeutic endpoints for shoulder pain embodiments include a clinically meaningful reduction in patient pain scores as measured by the Visual Analog Scale (VAS) or Numeric Rating Scale (NRS), and improvement in shoulder function as measured by validated outcome instruments including the Shoulder Pain and Disability Index (SPADI), the Oxford Shoulder Score (OSS), or the American Shoulder and Elbow Surgeons (ASES) standardized shoulder assessment form. In some embodiments, the average decrease in patient-reported shoulder pain scores is at least 30% from baseline.

### A Use Case Scenario for Treating Lateral Epicondylitis (Tennis Elbow) and Elbow Tendinopathy

Lateral epicondylitis, commonly referred to as tennis elbow, is a chronic pain condition caused by tendinopathy of the extensor carpi radialis brevis (ECRB) and associated extensor tendons at their origin on the lateral epicondyle of the humerus. The condition is characterized by pathological neovascularization and sensory nerve ingrowth into the tendon, producing refractory pain in a substantial proportion of patients who fail conservative management. Transcatheter arterial embolization (TAE) targeting the neovasculature of the lateral epicondyle via branches of the radial recurrent artery and posterior interosseous recurrent artery can be effective in patients with refractory lateral epicondylitis.

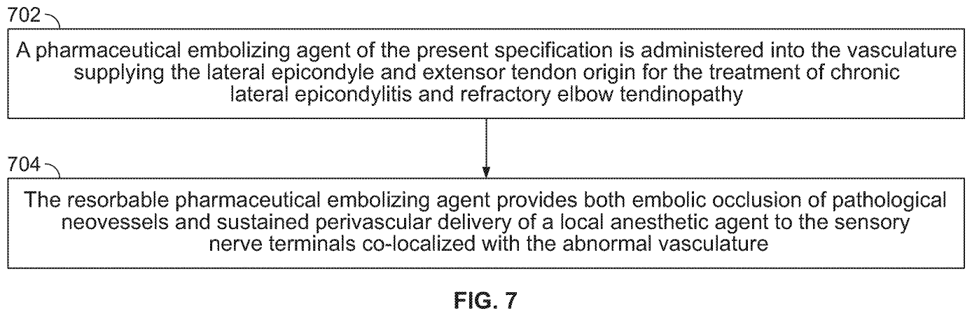

FIG. 7 is a flowchart describing exemplary steps of a method of treating lateral epicondylitis (Tennis Elbow) and elbow tendinopathy, in accordance with some embodiments of the present specification. At step 702, a pharmaceutical embolizing agent of the present specification is administered into the vasculature supplying the lateral epicondyle and extensor tendon origin for the treatment of chronic lateral epicondylitis and refractory elbow tendinopathy.

The primary vascular targets include branches of the radial recurrent artery and the posterior interosseous recurrent artery that supply the peritendinous and intratendinous pathological neovasculature at the lateral epicondyle.

In some embodiments, the pharmaceutical embolizing agent comprising the second composition (medical gel with sustained-release bupivacaine or other long-acting local anesthetic) is used in this setting.

At step 704, the resorbable pharmaceutical embolizing agent provides both embolic occlusion of pathological neovessels and sustained perivascular delivery of a local anesthetic agent to the sensory nerve terminals co-localized with the abnormal vasculature.

Therapeutic endpoints for lateral epicondylitis embodiments include clinically meaningful reduction in elbow pain on the Patient-Rated Tennis Elbow Evaluation (PRTEE) score, or equivalent validated instrument, from baseline. In some embodiments, the average decrease in patient-reported elbow pain scores is at least 30% from pre-treatment baseline.

### A Use Case Scenario for Treating Chronic Hip Pain and Hip Osteoarthritis

Chronic hip pain arising from osteoarthritis, labral pathology, or periarticular soft tissue conditions represents a significant source of musculoskeletal disability. The pathophysiology involves synovial inflammation, subchondral bone neovascularization, and abnormal neovascular ingrowth into the articular cartilage margin, mediated in part by VEGF-driven angiogenesis. Transcatheter arterial embolization of the hip's periarticular vasculature has been described in early clinical case series as a potential treatment for refractory hip pain.

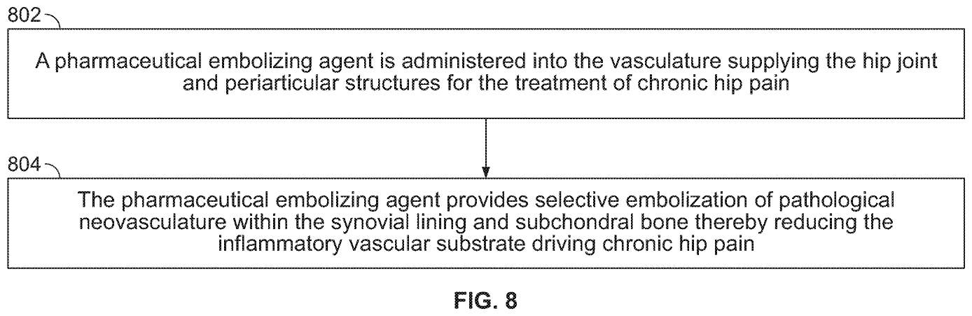

FIG. 8 is a flowchart describing exemplary steps of a method of treating chronic hip pain and hip osteoarthritis, in accordance with some embodiments of the present specification. At step 802, a pharmaceutical embolizing agent is administered into the vasculature supplying the hip joint and periarticular structures for the treatment of chronic hip pain. The primary arterial targets include branches of the medial circumflex femoral artery, which is the dominant supply to the femoral head and hip joint capsule; branches of the lateral circumflex femoral artery supplying the anterior capsule and periarticular soft tissues; and the inferior gluteal artery supplying the posterior capsule and periarticular structures.

In some embodiments, the pharmaceutical embolizing agent comprising the second composition (medical gel with sustained-release bupivacaine or other long-acting local anesthetic) is used in this setting.

At step 804, the pharmaceutical embolizing agent provides selective embolization of pathological neovasculature within the synovial lining and subchondral bone thereby reducing the inflammatory vascular substrate driving chronic hip pain.

In embodiments for hip pain, either the first composition (medical gel with anti-VEGF agent) or the second composition (medical gel with bupivacaine) may be employed. The first composition targets pathological VEGF-driven synovial neovascularization; the second composition additionally provides sustained perivascular anesthetic delivery. Therapeutic endpoints include clinically meaningful improvement on the Hip disability and Osteoarthritis Outcome Score (HOOS) or the Oxford Hip Score (OHS). In some embodiments, the average decrease in patient-reported hip pain scores is at least 30% from baseline.

### A Use Case Scenario for Treating Plantar Fasciitis and Chronic Heel Pain

Plantar fasciitis is the most common cause of heel pain, affecting a substantial proportion of the adult population. Chronic plantar fasciitis is characterized by pathological neovascularization at the calcaneal enthesis of the plantar fascia, with co-localized sensory nerve ingrowth, consistent with the same pathological mechanism as rotator cuff tendinopathy and lateral epicondylitis. Transcatheter arterial embolization targeting the pathological neovasculature of the plantar fascia calcaneal attachment can be effective in patients with refractory plantar fasciitis.

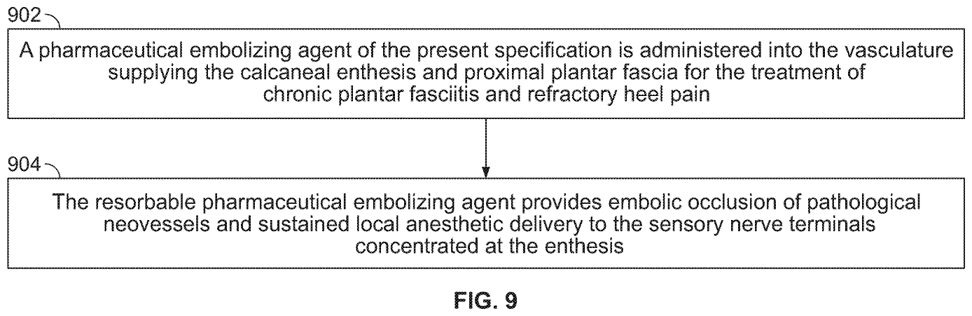

FIG. 9 is a flowchart describing exemplary steps of a method of treating plantar fasciitis and heel pain, in accordance with some embodiments of the present specification. At step 902, a pharmaceutical embolizing agent of the present specification is administered into the vasculature supplying the calcaneal enthesis and proximal plantar fascia for the treatment of chronic plantar fasciitis and refractory heel pain.

In some embodiments, the pharmaceutical embolizing agent comprising the second composition (medical gel with sustained-release bupivacaine or other long-acting local anesthetic) is used in this setting.

The primary vascular targets include branches of the medial plantar artery and lateral plantar artery (terminal divisions of the posterior tibial artery) supplying the plantar fascia origin, the calcaneal branches of the posterior tibial artery supplying the calcaneal enthesis, and, where neovascularization is present within the plantar fascia body, the accompanying intratendinous neovessels.

At step 904, the resorbable pharmaceutical embolizing agent provides embolic occlusion of pathological neovessels and sustained local anesthetic delivery to the sensory nerve terminals concentrated at the enthesis.

Therapeutic endpoints for plantar fasciitis embodiments include clinically meaningful reduction in heel pain on the Visual Analog Scale (VAS) or the Foot and Ankle Ability Measure (FAAM), and improvement in functional walking capacity. In some embodiments, the average decrease in patient-reported heel pain scores is at least 30% from baseline.

### A Use Case Scenario for Treating Discogenic Low Back Pain Via Basi Vertebral Artery Embolization

Discogenic low back pain arising from vertebral endplate pathology and activation of the basivertebral nerve (BVN) is recognized as a major contributor to chronic low back pain. The basivertebral nerve is an intraosseous nerve within the vertebral body that innervates the endplates and is activated by inflammatory and mechanical stimuli from degenerate intervertebral discs. The basivertebral nerve is supplied by the basivertebral artery, an intraosseous branch of the posterior vertebral body arteries arising from the segmental lumbar arteries. Radiofrequency ablation of the basivertebral nerve via transpedicular approach has received regulatory clearance as a treatment for chronic vertebrogenic low back pain, validating the basivertebral nerve as a therapeutic target.

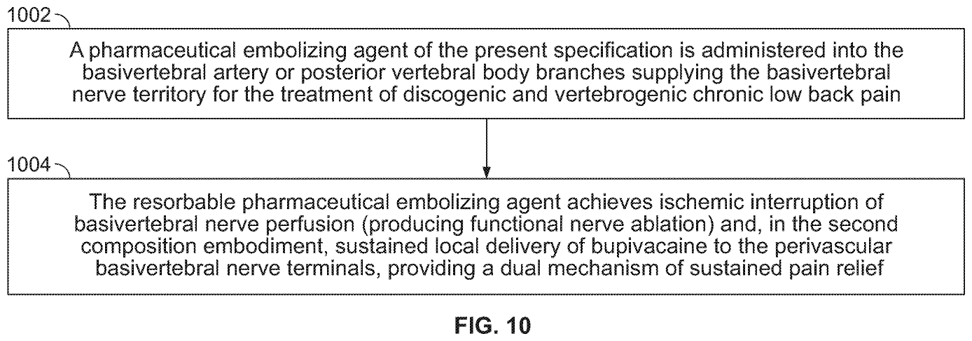

FIG. 10 is a flowchart describing exemplary steps of a method of treating discogenic low back pain, in accordance with some embodiments of the present specification. At step 102, a pharmaceutical embolizing agent of the present specification is administered into the basivertebral artery or posterior vertebral body branches supplying the basivertebral nerve territory for the treatment of discogenic and vertebrogenic chronic low back pain.

This transarterial approach constitutes a less invasive alternative to transpedicular radiofrequency ablation for the same therapeutic target: unlike transpedicular needle-based procedures, intra-arterial delivery does not require bony penetration of the pedicle and can be performed via standard endovascular microcatheter technique.

In some embodiments, the pharmaceutical embolizing agent is administered at one or more vertebral levels (typically L3-S1) corresponding to the Modic change pattern or MRI signal change pattern in the vertebral endplates that is indicative of basivertebral nerve activation and vertebrogenic pain.

In some embodiments, the pharmaceutical embolizing agent comprising the second composition (medical gel with sustained-release bupivacaine or other long-acting local anesthetic) is used in this setting.

At step 1004, the resorbable pharmaceutical embolizing agent achieves at least one of ischemic interruption of basivertebral nerve perfusion (producing functional nerve ablation) and sustained local delivery of bupivacaine to the perivascular basivertebral nerve terminals, providing a dual mechanism of sustained pain relief.

Therapeutic endpoints include clinically meaningful improvement on the Oswestry Disability Index (ODI), with a minimum clinically important difference (MCID) of at least 10 points, or equivalent validated outcome measure for chronic low back pain. In some embodiments, the average decrease in patient-reported low back pain scores is at least 30% from baseline at 12 weeks post-treatment.

### A Use Case Scenario for Treating Facet-Mediated Spinal Pain Via Medial Branch Artery Embolization

Facet joint-mediated pain (zygapophyseal joint pain) is estimated to contribute to 30-40% of chronic axial low back pain, with comparable prevalence in the cervical spine. The facet joint and its capsule are innervated by the medial branch of the dorsal ramus of the spinal nerve, which also supplies the multifidus muscle and interspinous structures. Medial branch nerve blocks and radiofrequency neurotomy of the medial branch are established standard-of-care interventional procedures for facet-mediated pain, confirming the medial branch as a validated analgesic target.

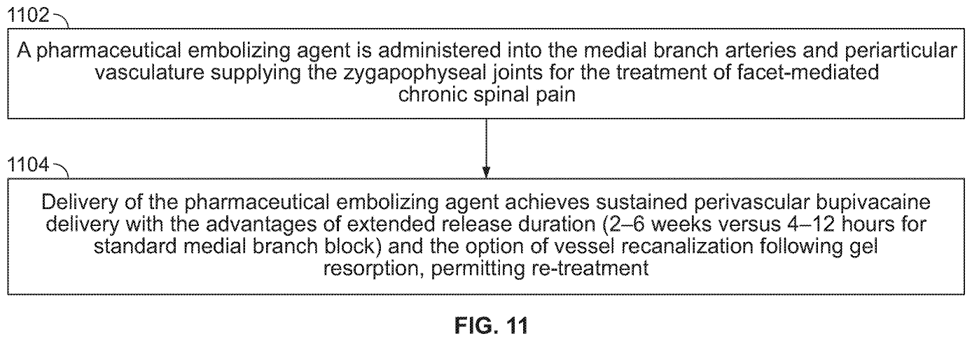

FIG. 11 is a flowchart describing exemplary steps of a method of treating facet-mediated pain, in accordance with some embodiments of the present specification. At step 1102, a pharmaceutical embolizing agent is administered into the medial branch arteries and periarticular vasculature supplying the zygapophyseal joints for the treatment of facet-mediated chronic spinal pain.

The medial branch arteries arise from the dorsal branch of the segmental lumbar arteries and supply the facet joint capsule, the medial branch nerve, and the multifidus muscle. Intra-arterial delivery of the second composition to these vessels achieves sustained perivascular bupivacaine delivery to the medial branch nerve terminals, providing a transarterial equivalent of medial branch block with the advantages of extended-release duration (2-6 weeks versus 4-12 hours for standard medial branch block) and the option of vessel recanalization following gel resorption, permitting re-treatment.

In embodiments for cervical facet pain, the pharmaceutical embolizing agent is administered into the medial branch arteries arising from the deep cervical or vertebral artery branches supplying the cervical zygapophyseal joints (C2-C7). In embodiments for lumbar facet pain, the pharmaceutical embolizing agent is administered into medial branch arteries arising from the dorsal branches of the L1-L5 segmental lumbar arteries, targeting one or more lumbar levels according to the clinical pain pattern.

At step 1104, delivery of the pharmaceutical embolizing agent achieves sustained perivascular bupivacaine delivery with the advantages of extended-release duration (2-6 weeks versus 4-12 hours for standard medial branch block) and the option of vessel recanalization following gel resorption, permitting retreatment. In embodiments, the potential for a repeat treatment depends on the patient's symptoms and/or clinical judgment.

### A Use Case Scenario for Treating Craniofacial Pain Via Sphenopalatine Artery Embolization

The sphenopalatine ganglion (SPG) is a parasympathetic ganglion located in the pterygopalatine fossa that plays a central role in the pathophysiology of cluster headache, trigeminal neuralgia, and other craniofacial pain syndromes. The SPG receives arterial supply primarily from the sphenopalatine artery and pterygopalatine branches of the internal maxillary artery, which is accessed via the external carotid artery. Existing SPG-targeted therapies include intranasal lidocaine delivery, percutaneous SPG block, and SPG radiofrequency ablation, confirming the SPG as a validated analgesic target for refractory craniofacial pain.

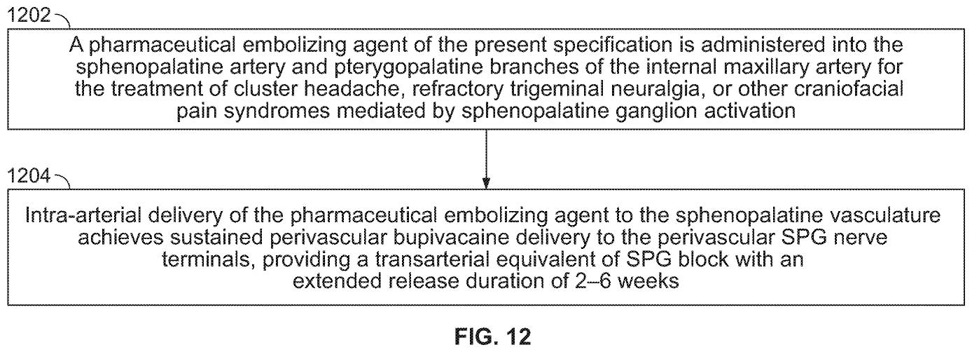

FIG. 12 is a flowchart describing exemplary steps of a method of treating craniofacial pain, in accordance with some embodiments of the present specification. At step 1202, a pharmaceutical embolizing agent of the present specification is administered into the sphenopalatine artery and pterygopalatine branches of the internal maxillary artery for the treatment of cluster headache, refractory trigeminal neuralgia, or other craniofacial pain syndromes mediated by sphenopalatine ganglion activation.

In some embodiments, the pharmaceutical embolizing agent comprising the second composition (medical gel with sustained-release bupivacaine or other long-acting local anesthetic) is used in this setting.

At step 1204, intra-arterial delivery of the pharmaceutical embolizing agent to the sphenopalatine vasculature achieves sustained perivascular bupivacaine delivery to the perivascular SPG nerve terminals, providing a transarterial equivalent of SPG block with an extended-release duration of 2-6 weeks. The resorbable nature of the pharmaceutical embolizing agent allows vessel recanalization following gel degradation, permitting re-treatment.

Therapeutic endpoints for craniofacial pain embodiments include clinically meaningful reduction in headache frequency and severity as measured by validated instruments including the Headache Impact Test (HIT-6), Migraine Disability Assessment (MIDAS), or the Cluster Headache Disability Scale (CHDS). In some embodiments, the average decrease in patient-reported craniofacial pain scores is at least 30% from baseline.

### A Use Case Scenario for Treating Occipital Neuralgia

Occipital neuralgia is a chronic pain syndrome characterized by paroxysmal shooting or stabbing pain in the distribution of the greater occipital nerve (GON) or lesser occipital nerve (LON), arising from irritation or entrapment of these nerves in the suboccipital region. The occipital artery provides the primary blood supply to the suboccipital musculature, the greater and lesser occipital nerves, and the posterior scalp. Occipital nerve blocks and pulsed radiofrequency neuromodulation of the occipital nerve are established treatments for refractory occipital neuralgia.

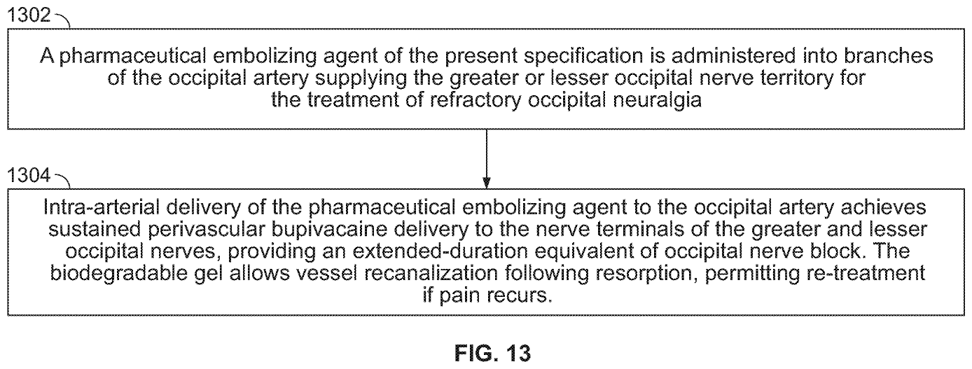

FIG. 13 is a flowchart describing exemplary steps of a method of treating occipital neuralgia, in accordance with some embodiments of the present specification. At step 1302, a pharmaceutical embolizing agent of the present specification is administered into branches of the occipital artery supplying the greater or lesser occipital nerve territory for the treatment of refractory occipital neuralgia.

In some embodiments, the pharmaceutical embolizing agent comprising the second composition (medical gel with sustained-release bupivacaine or other long-acting local anesthetic) is used in this setting.

At step 1304, intra-arterial delivery of the pharmaceutical embolizing agent to the occipital artery achieves sustained perivascular bupivacaine delivery to the nerve terminals of the greater and lesser occipital nerves, providing an extended-duration equivalent of occipital nerve block. The biodegradable gel allows vessel recanalization following resorption, permitting re-treatment if pain recurs. In embodiments, the potential for a repeat treatment depends on the patient's symptoms and/or clinical judgment.

The above examples are merely illustrative of the many applications of the systems and methods of the present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method of treating pain in a patient, the method comprising:
forming a vascular access into the patient;
advancing a catheter through the patient's vasculature to a target artery supplying blood to a pain-associated anatomical region; and
administering through the catheter a pharmaceutical embolizing agent to a location in said target artery, wherein the pharmaceutical embolizing agent comprises an injectable biocompatible medical gel and a sustained-release analgesic incorporated within the medical gel, wherein the pharmaceutical embolizing agent is adapted to (i) form an embolus at said location, (ii) release a therapeutic drug at said location over time, and (iii) be structurally stabilized at the location to retain the analgesic without migration within the target artery and release the analgesic over an extended period to reduce pain experienced by the patient.

2. The method of claim 1, further comprising administering the pharmaceutical embolizing agent by injection such that the analgesic is released at least 5 cm beyond the patient's main middle meningeal artery trunk into distal meningeal branches to thereby achieve perivascular contact between the analgesic and trigeminal afferents innervating the patient's distal meningeal territory.

3. The method of claim 1, wherein the sustained-release analgesic comprises bupivacaine and functional equivalents thereof.

4. The method of claim 1, wherein the sustained-release analgesic is encapsulated within liposomes, microspheres, or nanoparticles dispersed in the medical gel.

5. The method of claim 1, wherein the pharmaceutical embolizing agent is configured to fully release all of the analgesic over a period of at least 2 weeks and less than 10 weeks following administration.

6. The method of claim 1, wherein the medical gel comprises a hydrogel comprising at least one of polyethylene glycol hydrogels, chitosan hydrogels, alginate hydrogels, fibrin hydrogels, hyaluronic acid hydrogels, and PLGA-based gels.

7. The method of claim 1, wherein the pharmaceutical embolizing agent exhibits shear-thinning rheological characteristics enabling delivery through a microcatheter and structural recovery after deposition within the target artery.

8. The method of claim 1, wherein the pharmaceutical embolizing agent has a viscosity between approximately 50 and 500 centipoise during injection.

9. The method of claim 1, wherein the pharmaceutical embolizing agent is biodegradable and non-permanent, allowing recanalization of the target artery over time.

10. The method of claim 1, wherein the pharmaceutical embolizing agent further comprises a radiopaque material enabling visualization under fluoroscopic imaging.

11. The method of claim 10, wherein the radiopaque material comprises at least one of tantalum, tungsten, barium, and polymer-bound iodine.

12. The method of claim 1, wherein administering the pharmaceutical embolizing agent comprises injecting the agent through a microcatheter having an inner diameter between approximately 0.0135 and 0.0314 inches.

13. The method of claim 1, wherein administering the pharmaceutical embolizing agent comprises injecting the pharmaceutical embolizing agent at a rate between approximately 0.01 mL/s and 0.05 mL/s.

14. The method of claim 1, wherein the target artery comprises a middle meningeal artery.

15. The method of claim 1, wherein the pain comprises at least one of headache pain, migraine pain, post-craniotomy pain, post-operative pain, spine pain, knee pain, and low back pain associated with spinal structures supplied by lumbar arterial branches.

16. The method of claim 1, wherein an amount of the analgesic is less than approximately 10% by weight of the pharmaceutical embolizing agent.

17. The method of claim 1, wherein the location is defined by a region extending 0.1 cm to 25 cm into the target artery from an injection site.

18. The method of claim 1, wherein, after said administration, the patient experiences a reduction of at least 30% in a pain score measured using a standardized pain assessment scale.

19. The method of claim 1, wherein the pharmaceutical embolizing agent exhibits at least one of a power-law flow behavior index between approximately 0.3 and 0.4 and a viscosity between 50 and 500 cP at shear rates of 0.1-10 s⁻¹.

20. The method of claim 1, wherein, once administered at the location, the medical gel has an elastic modulus (G′) of 10 to 500 Pascals, such that the administered medical gel deforms compatibly with physiological arterial wall pulsation.
